(19) United States
(12) Patent Application Publication
(10) Pub. No.: US 2019/0172557 A1
(43) Pub. Date: Jun. 6, 2019

(54) Rule-Based System to Detect Metastatic Cancer Stemming from a Colorectal Tumor and to Determine a Proposed Treatment Regime

(71) Applicant: Persona Biomed, Inc., Alexandria, VA (US)

(72) Inventor: Fernando Vidal-Vanaclocha, Alexandria, MD (US)

(73) Assignee: Persona Biomed, Inc., Alexandria, VA (US)

(21) Appl. No.: 16/210,486

(22) Filed: Dec. 5, 2018

Related U.S. Application Data
(60) Provisional application No. 62/595,004, filed on Dec. 5, 2017.
(60) Provisional application No. 62/596,870, filed on Dec. 10, 2017.

(51) Int. Cl. G16B 40/00; G16B 30/00

(52) U.S. Cl. CPC G16B 40/00; G16B 30/00

(57) ABSTRACT

Rule-based apparatus and kit to detect metastatic cancer in a patient having a colorectal tumor. Apparatus has inputs to receive patient data comprising expression levels of genes from hepatic tissue, a memory that stores reference values of corresponding genes of persons free of colorectal tumors, a processor that interprets significance of overexpression and/or underexpression of selected patient data genes relative to reference values, and an output responsive to the processor to produce an indication confirming or annulling hepatic metastasis. Specific genes in questions include group 1 genes (PRDX4, CRP, ID1, MT1E, TNFSF14, MRC1, ICAM1, IL18, IL10, TFN) and group 2 genes (NGF, EPHA1, ERBB2IP, SDC1, COL18A1, KNG1, ADH1 B, CYP2E1). Analysis may be performed on protein signatures of genes rather than genes themselves. A kit to assist a user to produce patient data comprises a low density genetic expression array or protein array for measuring genetic expressions or protein signature, as well as instructions for using the kit.

### CROSS-REFERENCE TO RELATED PATENT APPLICATIONS

This invention claims the benefit of U.S. Provisional Application Ser. No. 62/595,004 filed Dec. 5, 2017 in the name of the same inventor hereof, and entitled Method of Diagnosing and Treating Colorectal Cancer Based on Divergent Liver Prometastatic Gene Expression Patterns and U.S. Provisional Application Ser. No. 62/596,870 filed Dec. 10, 2017 in the name of the same inventor hereof and entitled Method of Processing Liver Prometastatic Gene Expression Patterns in a Rule-Based Diagnostic and Treatment Determination System, the disclosure of each of which is incorporated herein by reference.

This application is related to U.S. Nonprovisional application Ser. No. 16/209,521, filed Dec. 4, 2018 in the name of the same inventor hereof and entitled Method of Detecting and/or Treating Colorectal Cancer Based on Divergent Liver Prometastatic Gene Expression Patterns, which is incorporated herein.

### BACKGROUND

This invention concerns a rule-based system and kit useful for detecting metastatic cancer or risk thereof in a patient having a colorectal tumor, as well as an automated system to determine a proposed treatment regime. The system or apparatus uses inputs derived from detection of divergent liver prometastatic gene expression patterns that occur in the tumor microenvironment. In addition, protein signatures of involved genes rather than genes themselves may be used as inputs where such protein signature are derived from analysis of the patient's blood serum or plasma.

The liver is a major metastasis-susceptible site in the human body and a majority of patients with hepatic metastases die from the disease regardless of treatment. Presently, hepatic metastasis is conventionally detected by imaging techniques, which typically cannot detect cancer lesions less than about five to seven millimeters in diameter. By the time the lesion reaches that size, however, millions or even billion of cancer cells have already spread throughout the patient's body and little if anything can be done to abate the disease. Thus, the average CRC (colorectal cancer) patient dies within two to five years, more or less.

A focal liver lesion in the liver, for example, more likely represents a metastatic tumor than a primary malignancy. In addition, a majority of patients develop multiple liver metastases in both lobes that vary in diameter suggesting that cancer cell seeding and growth occur in independent and separate episodes. Numerous experimental and clinical studies have focused on factors that regulate metastasis recurrence in the liver. At present, however, genetic and phenotypic properties of specific cancer cells able to implant and grow in the liver have not yet been established for any primary tumor type. Neither the contribution of the patient's genetic expressions nor the patient's physiologic background to the incidence and progression of hepatic metastases is presently understood.

Liver metastasis development is promoted by a broad range of organ-specific prometastatic factors, including cancer cell growth-stimulating factors, tumor stromal cell-stimulating factors, tumor angiogenesis-stimulating factors, and hepatic immune suppressant factors, among others. The experimental identification of some of these factors made it possible to understand certain hepatic metastasis development inhibition (Vidal-Vanaclocha F. The prometastatic microenvironment of the liver. Cancer Microenvironment, 2008;1:113-129). However, it is not clear if these diverse factors have a control role during human liver metastasis disease. Neither is it clear if such factors have already occurred prior to CRC development (as a constitutive predisposition to liver metastasis), if they were induced by certain comorbidities and therapies, and/or if they were induced remotely by CRC cells endowed with this prometastatic feature.

Therefore, it is plausible that the liver might acquire a prometastatic condition concomitant with CRC progression and that such condition might be activated by either tumor-dependent or tumor-independent factors. Either way, these factors may activate remotely a “Liver Prometastatic Reaction” (LPR) favorable for the hepatic colonization of circulating cancer cells, and they should be designated as LPR-stimulating factors (LPR-SF), irrespective of their nature.

Production of LPR-SF and their delivery into the mesenteric vein circulation may be upregulated in CRC cells (including tumor and non-tumor stromal cell lineages) by tumor site-dependent factors (as for example, colonic inflammation, tumor hypoxia and mechanical stress, diet, gut microflora-derived bacterial factors, etc.), but also by factors from other intraperitoneal organs whose venous blood is draining into the mesenteric veins (spleen, pancreas, visceral fat, etc.). In addition, they may also be activated by systemic factors reaching the liver through the hepatic artery.

Once developed, the LPR may in turn lead to the hepatic cell production of Metastasis-Stimulating Factors (LPR-derived MSF) of potential interest as targets for anti-metastatic therapy. Their specific hepatic cell origin and their nature and effects on both cancer and stromal cells are now being recently understood. For example, LPR-derived MSF upregulated CRC cell expression of certain liver metastasis-specific genes, not expressed at primary CRC, suggesting they may also represent liver metastasis-specific molecular targets for therapy.

Therefore, LPR-specific genes and proteins may represent clinically-valuable hepatic biomarkers for predicting a risk level and/or detecting development of hepatic CRC metastasis. In addition, LPR-derived soluble factors should leave the liver through the suprahepatic vein and therefore they should be detectable in the peripheral blood, alerting on the occurrence of LPR in a given cancer patient.

The possibility that LPR-derived MSF can regulate some of liver metastasis-associated genes suggests that the CRC prometastatic phenotype includes both liver-independent and liver-dependent metastasis-associated genes, the first occurring at the primary tumor and the second only at metastatic sites, activated by the LPR-derived MSF. Therefore, liver-independent metastasis-associated CRC genes may have diagnostic value as prometastatic detectors or predictors at the primary tumor, while liver-dependent metastasis-associated CRC genes, which should be detectable at metastatic, but not at primary sites, may be valuable as targets for therapy. In addition, liver-independent metastasis-associated CRC genes may be involved in the CRC production of LPR-SF, which in turn would induce LPR-derived MSF further supporting hepatic metastasis development.

The inventor hereof has discovered that development of hepatic metastases is associated with an aberrant tissue-reconstitution process that results from bidirectional reciprocal effects between cancer cells and resident hepatic cells. On one hand, cancer cells and their soluble and exosomal proteins regulate gene expression in hepatic cells residing in, or infiltrating into, various sites of metastases. At these sites, cancer cells exert selective pressures on hepatic cells thereby shaping their functional phenotypes. Conversely, constituents of the liver microenvironment may also regulate gene expression in the cancer cells thereby controlling their fate and determining their ability to progress towards metastatic formation.

Additionally, there are pathophysiological processes such as aberrant hepatic regeneration, inflammation and fibrosis that change the hepatic microenvironment and notably affect development of metastases. Therefore, tumor microenvironment regulating hepatic metastasis in a given patient consists of structural and functional factors resulting from both hepatic-cancer cell interactions and previous or concurrent hepatic diseases.

Neoplasms from right and left colon and rectum frequently metastasize to the liver. At a transcriptional level, hepatic metastasis development is in part associated with marked changes in gene expression of colorectal cancer cells that may originate in a primary tumor. Other prometastatic changes occur in the liver and are regulated by hepatic cells, which represent a new microenvironment for metastatic colon cancer cells. In addition, hepatic parenchymal and non-parenchymal cell functions are also affected by both cancer cell-derived factors and various systemic pathophysiological factors of a patient having CRC.

Liver and gastrointestinal tract physiology and pathology are interrelated. For example, gallstones (cholelithiasis) and cholecystectomy are related to digestive system cancer through inflammation, altered bile flux, and changes in metabolic hormone levels. More importantly, it has been established that a statistically significant risk of colorectal cancer follows cholelithiasis (Lee et al, 2016; Gosavi et al, 2017). Similarly, fatty liver, which is a hepatic manifestation of metabolic syndrome, is a well-known risk factor for CRC (Barbois et al, 2017). If hepatic gene expression disorders precede CRC occurrence, early biomarkers of CRC risk and development may be assessed.

In the past two decades, a growing amount of data has been reported suggesting that carcinomas of the right and left colon should be considered as different tumor entities. Right-sided colon cancers (RCC) and left-sided colon cancers (LCC) are of different embryological origins, and various differences exist between them. Tumor location is associated with prognosis in colorectal cancer patients, and those with RCC have a significantly worse prognosis than those with LCC (Yahigi et al 2016). RCC should be treated distinctively from LCC (Zhao et al, 2017), and the establishment of standardized management for colon cancer by tumor location is needed.

Characterization of genes that are differentially expressed in tumorigenesis is an important step in identifying those that are intimately involved in the details of a cell's transformation from normal to cancerous, and from non-metastatic to metastatic cells. However, little is known about molecular changes that occur in key organs (as for example the liver) during the course of cancer development and its metastatic disease. While changes in the expression level of individual genes have been reported, investigation of gene expression changes that occur in the liver of patients with cancer and without cancer as provided by the present invention has not been previously known or documented.

In brief summary, there exists a need in the art for the identification of new CRC disease-associated hepatic genes and resultant proteins as molecular biomarkers to, among other things, to (i) monitor and assess the pathogenic contribution of liver to CRC and hepatic CRC metastasis development; (ii) identify and/or screen candidate cancer patients suitable for liver metastasis-specific therapies at the cancer microgenesis stage rather than by imaging; and (iii) discover and/or screen pharmaceutical cellular and molecular compositions targeting those liver genes with CRC and CRC metastasis-stimulating activities in patients with colorectal cancer or CRC risk thereof.

These and other needs are met by various aspects of the present invention.

### SUMMARY

According to a first aspect of the invention, there is provided a rule-based apparatus (a manually operated or digital device) to detect metastatic cancer in a patient having a colorectal tumor, where the apparatus comprises (a) an input to receive patient data comprising a plurality of genetic expression levels of genes from tumor unaffected hepatic tissue of the patient where the genes include selected ones of genes from group 1 genes (PRDX4, CRP, ID1, MT1E, TNFSF14, MRC1, ICAM1, IL18, IL10, TFN) and group 2 genes (NGF, EPHA1, ERBB2IP, SDC1, COL18A1, KNG1, ADH1B, CYP2E1), (b) a memory or gene database that stores respective reference values for group 1 and group 2 genes wherein the reference values respectively indicate expression levels of corresponding genes of a person free of colorectal tumors, (c) a processor responsive to patient data and reference values to interpret the significance of overexpression of selected group 1 patient data genes relative to group 1 reference values and/or significance of underexpression of selected group 2 patient data genes relative to group 2 reference values; and (d) an output responsive to said processor to produce an indication confirming or annulling hepatic metastasis according to interpretation of significance of overexpression and/or underexpression of group 1 and/or group 2 patient data genes.

Other aspects of the invention include wherein processor assigns a weight to respective genes according to predetermined significance of indication of hepatic metastasis; wherein the processor utilizes protein signatures of said genes to indicate overexpressions or underexpressions thereof; wherein the processor utilizes correlation, clustering and/or heatmaps to interpret significance of said gene expressions; wherein the processor additionally utilizes selected ones of group 3 genes (HP, VTN, RPS27, RPL23, GAPDH, TXN, VEGFA, CEACAM1, IGF1, TGFB1, DDR2, NOS2, and BMP7) to validate significance of gene express; wherein selected ones of genes comprising statistically significant ones of group 1, group 2 and group 3 genes; wherein the processor comprises a digital microprocessor instead of a manually operated device; and wherein the patient data and reference values comprise cycle count information derived from polymerase chain reactions, and;

A further aspect of the invention includes wherein the processor comprises a series of software modules to execute program instructions to perform at least two of (i) a partial least squares-discriminant analysis of said selected genes of a patient with and a patient without CRC, (ii) a clustering analysis of selected genes in a patient with and a patient without CRC, (iii) a Spearman's correlation analysis of selected genes to assess new and lost correlations of selected genes in respective categories in a patient with and a patient without CRC, (iv) a hierarchical clustering analysis of selected genes in a patient with and a patient without CRC, (v) distribution analysis of selected gene expression levels for genes in respective functional categories in a patient with and a patient without CRC, and (vi) determining high-low expression levels of selected genes in functional categories indicative of location of primary tumors in a patient with and a patient without CRC.

A yet further aspect of the invention includes wherein the processor detects (A) a left-side colon location of a CRC tumor according to overexpressed levels of statistically significant ones of (i) proinflammatory genes IL18, ID1, TNF, TNFSF14, AND ADH1B, (ii) immune regulation genes ICAM1, MRC1, KNG1, and SDC1, and/or (iii) metabolic bioprotection genes PRXD4, MTE1, P, NOS2 and CRP; (B) a rectal location of said CRC tumor according to underexpressed levels of statistically significant ones of (i) IL18, ID1, VEGFA, TNFSF14, ADH1B and CYP2E1 proinflammatory genes, (ii) ICAM1, KNG1, SDC1 AND BMP7 immuno regulation genes, and (iii) GAPDH, TXN, MTE1, HP, CR and ERBB2IP metabolic bioprotection genes; and (C) a right side colon location of said CRC tumor according to (i) high expression level of at least one of ID1 and TNF proinflammatory genes, (ii) low expression level of at least one of ADH18 and CYPE1 proinflammatory genes, (iii) high expression level of at least one of immune regulation genes IL10, MRC1 and BMP7, (iv) low expression level of at least one of immune regulation genes KNG1 and SDC1, and (v) low expression level of at least one of VTN and NGF fibrogenic and regeneration genes.

The invention further comprise a kit to derive patient data where the kit comprises (a) instructions for performing gene assessments and (b) either a low density genetic expression array of reagents for PCR replication/detection or a low density protein array of antibodies for hybridization with patient's blood serum/plasma;

Another aspect of the invention comprises a rule-based apparatus to detect occult cancer in a target patient having a gastrointestinal disorder, where the apparatus comprises (a) an input to receive patient data comprising a plurality of genetic expression levels of genes from tumor unaffected hepatic tissue of the patient, wherein the genes include selected ones of genes from group 1 (PRDX4, CRP, ID1, MT1E, TNFSF14, MRC1, ICAM1, IL18, IL10, TFN) and/or group 2 (NGF, EPHA1, ERBB2IP, SDC1, COL18A1, KNG1, ADH1B, CYP2E1); (b) a memory or database that stores respective reference values for group 1 and group 2 genes wherein the reference values indicate respective expression levels of corresponding genes of a person free of colorectal tumors; (c) a processor or computational device to receive and respond to patient data and the reference values (i) to interpret significance of overexpression of selected group 1 patient data genes relative to group 1 reference values and/or underexpression of selected group 2 patient data genes relative to group 2 reference values; and (d) an output responsive to said processor to produce an indication confirming or denying hepatic metastasis according to interpretation of significance of overexpression and/or underexpression of group 1 and/or group 2 patient data genes. This aspect of the invention may further include wherein the processor additionally utilizes selected ones of group 3 genes (HP, VTN, RPS27, RPL23, GAPDH, TXN, VEGFA, CEACAM1, IGF1, TGFB1, DDR2, NOS2, and BMP7) to validate significance of gene expression.

The invention also comprises a kit to assist a user to produce patient data for detecting hepatic metastasis where the kit comprises a low density genetic expression array for measuring genetic expressions of group 1 genes (PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1B, CYP2E1), and group 3 genes (HP, VTN, RPS27, RPL23, GAPDH, TXN, VEGFA, CEACAM1, IGF1, TGFB1, DDR2, NOS2, and BMP7) and instructions for sing the genetic expression array. The kit may also comprise low density protein array for measuring protein signatures of group 1 genes (PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1B, CYP2E1), and group 3 genes (HP, VTN, RPS27, RPL23, GAPDH, TXN, VEGFA, CEACAM1, IGF1, TGFB1, DDR2, NOS2, and BMP7) and (b) instructions for sing the protein array. The kit either includes a low density genetic expression array of reagents for PCR replication/detection or a low density protein array of antibodies for hybridization with patient's blood serum/plasma.

These and other aspects of the invention will become apparent upon review of the following disclosure taken in connection with the accompanying drawings. The invention, though, is pointed out with particularity by the appended claims.

### BRIEF DESCRIPTION OF THE DRAWINGS

FIGS. 1A and 1B show comparative transcriptomic analysis among metastatic CRC tissue, tumor-unaffected hepatic tissue and peripheral blood mononuclear cells from stage IV patients with CRC having systemic metastasis disease where FIG. 1A illustrates hybridization between RNA from metastatic CRC tissue and tumor-unaffected hepatic tissue and FIG. 1B illustrates hybridization between RNA from metastatic CRC tissue and peripheral blood mononuclear cells.

FIG. 1C shows a combination of FIGS. 1A and 1B in a single analysis diagram.

FIG. 1D is a Venn diagram showing overlapping sets of the number of genes for each of the three samples where CMN indicates mononuclear cells, M represent metastatic cells, and H represents tumor unaffected hepatic tissue.

FIG. 2 shows a logarithmic scale representation of the relative quantification (RQ) values of the liver prometastatic gene expression in CRC patients with respect to same values in patients without CRC.

FIG. 3 shows divergent liver prometastatic gene expression patterns in patients with and without CRC.

FIGS. 4A-4D respectively show distribution of liver prometastatic gene high-expressing patients by functional categories (proinflammatory, immune-regulation, metabolic Bioprotection and fibrogenic/regeneration) and tumor location in patients with CRC.

FIGS. 5A-5H show comparisons between proinflammatory gene expression levels in liver from patients with and without CRC.

FIGS. 6A-6I show comparisons between immuno-regulation gene expression levels in liver from patients with and without CRC.

FIGS. 7A-7H show comparisons between metabolic bioprotection gene expression levels in liver from patients with and without CRC.

FIGS. 8A-8F show comparisons between Fibrogenic and Regeneration gene expression levels in liver from patients with and without CRC.

FIG. 9 show comparison of overall expression profiles across samples from patients with and without CRC via a principal component analyses (PCA) of gene expressions in patients (P1-P51) with and in patients (C52-C72) without CRC where a first principal component (Dim 1) reveals a 27.18% of the variation and a second (Dim 2) reveals a 15.65% variation that separated most of patients with CRC from patients without CRC.

FIGS. 10A and 10B show Partial Least Squares-Discriminant Analysis (PLS-DA) intended to discriminate patients with and without CRC based on their hepatic expression level of liver prometastatic genes where elliptical shapes of FIG. 10A adopted by lines define the position coordinates of included patients (C indicates patients without CRC and P indicates patients with CRC), In this case, the discriminatory capacity was associated with the first component in the analysis. In FIG. 10B, position coordinates of liver prometastatic genes are plotted in correlation circles whose diameters define the influence of genes in the prediction of the class of patient where in this case metabolic bioprotection and fibrogenic/regeneration genes are in the smaller circle indicating that their expression levels had less ability to predict the patient's class than immune protection and proinflammatory genes, mainly located in the large correlation circle, and therefore had a greater predictive capacity to discriminate patients with and without CRC.

FIGS. 11A and 11B show heatmaps of clustering data for patients with and without CRC according their liver prometastatic gene expression patterns based on ΔΔCt ratios.

FIG. 12 shows Spearman's correlation patterns among liver prometastatic genes in patients with and without RCC.

FIGS. 13A-13D show hierarchical clustering was performed based on Pearson's correlation of Euclidean distance among the genes and gene clusters, and the results presented as a dendrogram plot in order to define the transcriptional structure of prometastatic genes in hepatic biopsies from patients without and with CRC (FIGS. 13A and 13C) and where a cluster including PRX4, SDC1, VEGFA, ID1 and CRP genes defined a main change in the hepatic transcriptional structure between patients without and with CRC (FIGS. 13B and 13D).

FIG. 14 is an exemplary functional block diagram of an apparatus to automate detection of hepatic metastasis based on inputs of gene expression or protein signatures thereof supplied by a user or by measurement/testing equipment where functions thereof may be carried out by programming instructions of a general purpose processor.

### DESCRIPTION OF ILLUSTRATIVE EMBODIMENTS

Disclosed herein are procedures and a device to detect occult CRC and liver metastasis and recurrence (i.e., a complementary diagnostic test) to identify candidate patients reasonably suitable to receive liver metastasis-specific therapies (a companion diagnostic test). The device uses, among other things, a series of mathematical, correlation and statistical analysis techniques to examine, compare and analyze relationships between and among expression levels of uniquely identified genes of hepatic tissues from patients with and without CRC. The invention includes utilization of a data processing device to automate gene analyses presented herein in order to provide a computer-determined output or result for diagnostic and/or treatment guidance to health care practitioners.

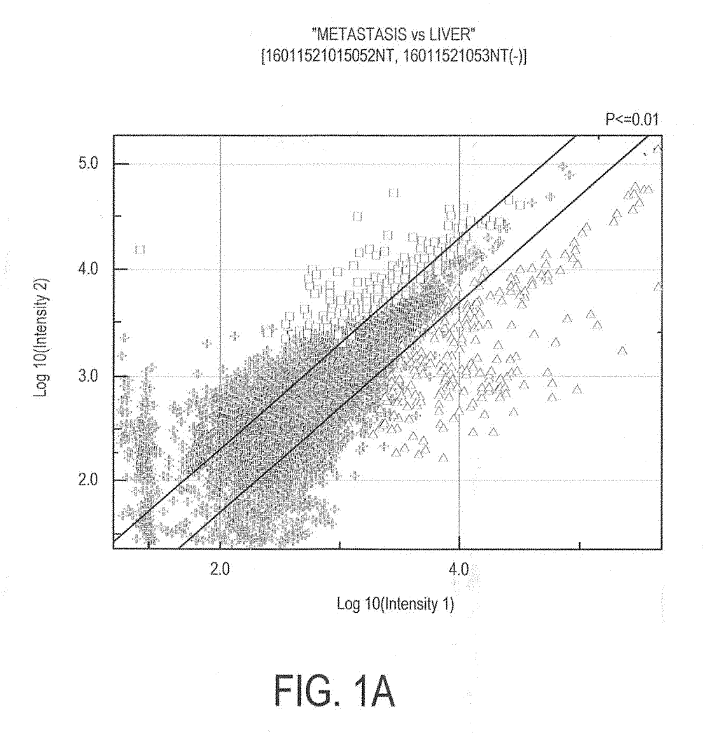

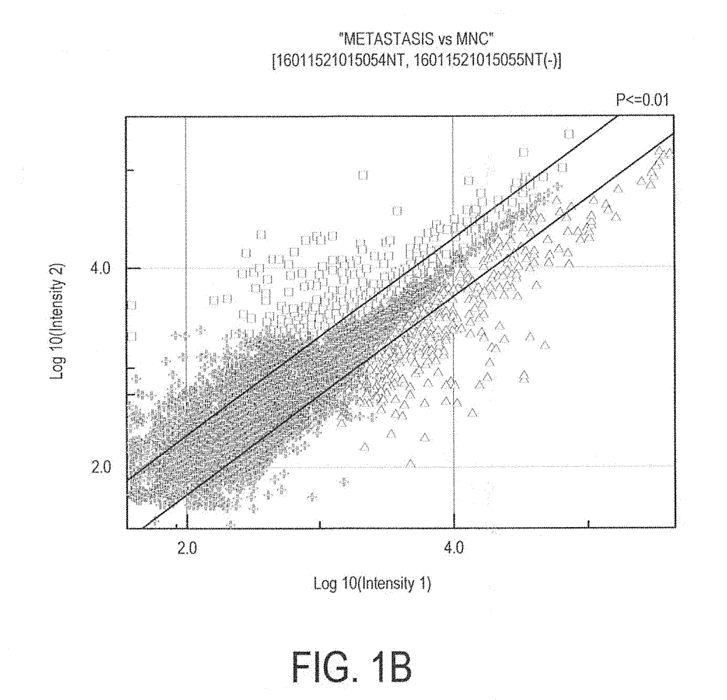

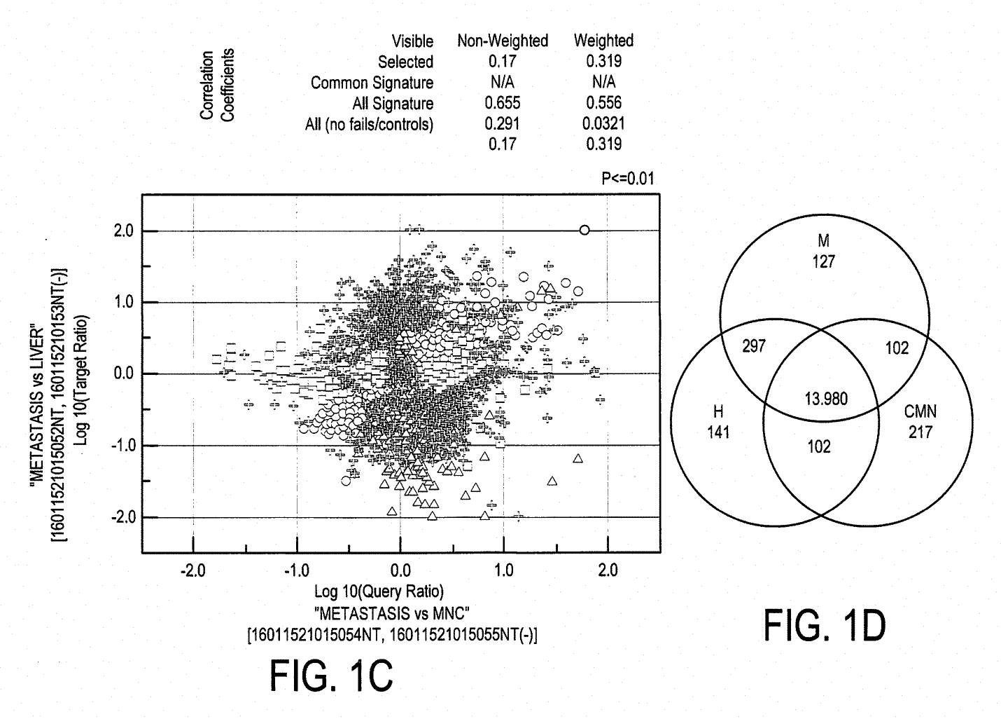

FIG. 1A shows a comparative transcriptomic analysis between metastatic CRC tissue and tumor-unaffected hepatic tissue from Stage IV patients with CRC having systemic metastasis disease. FIG. 1B shows a comparative transcriptomic analysis between metastatic CRC tissue and peripheral blood mononuclear cells from Stage IV patients with CRC having systemic metastasis disease. FIG. 1C combines that results of FIGS. 1A and 1B, while FIG. 1D shows the data in set and subset relation. These relationships help identify specific prometastatic genes used in the analysis.

More specifically, FIG. 1A shows hybridization between RNA from Metastatic CRC tissue and tumor-unaffected hepatic tissue; FIG. 1B shows hybridization between RNA from Metastatic CRC tissue and peripheral blood mononuclear cells; FIG. 1C shows a combination of FIGS. 1A and 1B; and FIG. 1D shows the number of genes for each sample compared and illustrated using a Venn diagram where “H” represents hepatic tissue, “M” represents metastatic tissue, and “CMN” represents mononuclear blood cells.

According to the analysis described in connection with FIGS. 1A, 1B, 1C and 1D, Table 1 below lists 122 genes whose expression levels were more than two-fold-upregulated in tumor-unaffected hepatic tissue compared to the expression levels in metastatic tissue and peripheral blood mononuclear cells from Stage IV patients with CRC having systemic metastasis disease.

In bold are twenty-one genes whose expression levels were upregulated in liver parenchymal and non-parenchymal sinusoidal cells given the conditioned medium from cultured CRC cells (HT-29 CRC cell line), This gene subset was selected for further analysis.

TABLE 1

| | Upregulated Genes |
|---|---|
| A1BG | Alpha-1-B glycoprotein |
| A2M | Alpha-2-macroglobulin |
| ABAT | 4-aminobutyrate aminotransferase |
| ACAA2 | Acetyl-Coenzyme A acyltransferase |
| ACAT1 | Acetyl-Coenzyme A acetyltransferase 1 |
| | (acetoacetyl Coenzyme A thiolase) |
| ADH1A | Alcohol dehydrogenase 1A (class I), alpha polypeptide |
| ADH1B | Alcohol dehydrogenase 1B (class I), beta polypeptide |
| ADH4 | Alcohol dehydrogenase 4 (class II), pi polypeptide |
| AFM | Afamin |
| AGT | Angiotensinogen (serpin peptidase |
| | inhibitor, clade A, member 8) |
| AHSG | Alpha-2-HS-glycoprotein (Fetuin A) |
| AKR1C2 | Aldo-keto reductase family 1, member C2 |
| ALAS1 | Aminolevulinate, delta-, synthase 1 |
| ALDOB | Aldolase B, fructose-bisphosphate |
| AMBP | Alpha-1-microglobulin/bikunin precursor |
| ANGPTL4 | Angiopoietin-like 4 |
| AOX1 | Aldehyde oxidase 1 |
| APOA2 | Apolipoprotein A-II |
| APOB | Apolipoprotein B (including Ag(x) antigen) |
| APOC1 | Apolipoprotein C-I |
| APOE | Apolipoprotein E |
| APOH | Apolipoprotein H (beta-2-glycoprotein I) |
| APOM | Apolipoprotein M |
| AQP9 | Aquaporin 9 |
| ARG1 | Arginase, liver |
| ASGR2 | Asialoglycoprotein receptor 2 |
| ATF5 | Activating transcription factor 5 |
| BBC3 | BCL2 binding component 3 |
| BRP44L | Brain protein 44-like |
| C1S | Complement component 1, s subcomponent |
| C3 | Complement component 3 |
| C4A | Complement component 4A (Rodgers blood group) |
| C4BPA | Complement component 4 binding protein, alpha |
| C8A | Complement component 8, alpha polypeptide |
| CBR1 | Carbonyl reductase 1 |
| CFB | Complement factor B |
| CFH | Complement factor H |
| CFHR1 | Complement factor H-related 1 |
| CFHR2 | Complement factor H-related 2 |
| CFI | Complement factor I |
| CLU | Clusterin |
| CP | Ceruloplasmin (ferroxidase) |
| CPS1 | Carbamoyl-phosphate synthetase 1, mitochondrial |
| CRP | C-reactive protein, pentraxin-related |
| CYB5A | Cytochrome b5 type A (microsomal) |
| CYP2C9 | Cytochrome P450, family 2, subfamily C, polypeptide 9 |
| CYP2E1 | Cytochrome P450, family 2, subfamily E, |
| | polypeptide 1 |
| CYP3A5 | Cytochrome P450, family 3, subfamily A, |
| | polypeptide 5 |
| DDR2 | Discoidin domain receptor tyrosine kinase 2 |
| EPHA1 | EPH receptor A1 |
| ERRFI1 | ERBB receptor feedback inhibitor 1 |
| F11 | Coagulation factor XI (plasma thromboplastin |
| | antecedent) |
| F12 | Coagulation factor XII (Hageman factor) |
| F2 | Coagulation factor II (thrombin) |
| F9 | Coagulation factor IX |
| FGA | Fibrinogen alpha chain |
| FGB | Fibrinogen beta chain |
| FGG | Fibrinogen gamma chain |
| FGL1 | Fibrinogen-like 1 |
| FOS | V-fos FBJ murine osteosarcoma viral oncogene |
| | homolog |
| FTCD | Formiminotransferase cyclodeaminase |
| GABARAPL3 | GABA(A) receptors associated protein like 3 |
| GAPDH | Glyceraldehyde-3-phosphate dehydrogenase |
| GC | Group-specific component (vitamin D binding protein) |
| GSTA2 | Glutathione S-transferase A2 |
| HAGH | Hydroxyacylglutathione hydrolase |
| HAMP | Hepcidin antimicrobial peptide |
| HP | Haptoglobin |
| HPN | Hepsin (transmembrane protease, serine 1) |
| HPX | Hemopexin |
| HSD11B1 | Hydroxysteroid (11-beta) dehydrogenase 1 |
| ICAM1 | Intercellular adhesion molecule-1 |
| ID1 | Inhibitor of differentiation-1 |
| IL10 | Interleukin-10 |
| IL18 | Interleukin-18 |
| ITIH1 | Inter-alpha (globulin) inhibitor H1 |
| ITIH4 | Inter-alpha (globulin) inhibitor H4 (plasma Kallikrein- |
| | sensitive glycoprotein) |
| KCNK7 | Potassium channel, subfamily K, member 7 |
| KNG1 | Kininogen 1 |
| LECT2 | Leukocyte cell-derived chemotaxin 2 |
| LPA | Lipoprotein, Lp(a) |
| LRP1 | Low density lipoprotein receptor-related protein 1 |
| MAT1A | Methionine adenosyltransferase I, alpha |
| MRC1 | Mannose receptor, C type 1 |
| MST1 | Macrophage stimulating 1 (hepatocyte growth factor-like) |
| MT1A | Metallothionein 1A |
| MT1B | Metallothionein 1B |
| MT1E | Metallothionein 1E |
| NR1I3 | Nuclear receptor subfamily 1, group I, member 3 |
| ORM1 | Orosomucoid 1 |
| ORM2 | Orosomucoid 2 |
| PCK2 | Phosphoenolpyruvate carboxykinase 2 (mitochondrial) |
| PLG | Plasminogen |
| PON3 | Paraoxonase 3 |
| POR | P450 (cytochrome) oxidoreductase |
| PRDX4 | Peroxiredoxin 4 |
| PXMP2 | Peroxisomal membrane protein 2, 22 kDa |
| PYROXD1 | Pyridine nucleotide-disulphide oxidoreductase domain 1 |
| RBP4 | Retinol binding protein 4, plasma |
| SAA4 | Serum amyloid A4, constitutive |
| SEPP1 | Selenoprotein P, plasma, 1 |
| SERPINA1 | Serpin peptidase inhibitor, clade A, member 1 |
| SERPINA6 | Serpin peptidase inhibitor, clade A, member 6 |
| SERPINC1 | Serpin peptidase inhibitor, clade C (antithrombin), |
| | member 1 |
| SERPIND1 | Serpin peptidase inhibitor, clade D (heparin |
| | cofactor), member 1 |
| SLC13A2 | Solute carrier family 13, member 2 |
| SLC27A5 | Solute carrier family 27 (fatty acid transporter), |
| | member 5 |
| SPP2 | Secreted phosphoprotein 2, 24 kDa |
| TF | Transferrin |
| TGFB1 | Transforming growth factor, beta 1 |
| THNSL2 | Threonine synthase-like 2 (S. cerevisiae) |
| TM4SF4 | Transmembrane 4 L six family member 4 |
| TNFA | Tumor necrosis factor-alpha |
| TNFSF14 | Tumor necrosis factor (ligand) superfamily, |
| | member 14 |
| TSPAN9 | Tetraspanin 9 |
| TTR | Transthyretin (prealbumin, amyloidosis type I) |
| TXN | Thioredoxin |
| UGT1A6 | UDP glucuronosyltransferase 1 family, polypeptide A4 |
| UGT2B7 | UDP glucuronosyltransferase 2 family, polypeptide B7 |
| VEGFA | Vascular endothelial growth factor A |
| VTN | Vitronectin |
| ZGPAT | Zinc finger, CCCH-type with G patch domain |

Table 2 below lists twenty-eight genes whose expression levels were more than two-fold-downregulated in tumor-unaffected hepatic tissue compared to the expression in metastatic tissue and peripheral blood mononuclear cells from Stage IV patients with CRC having systemic metastasis disease. In bold are ten genes whose expression levels were downregulated in liver parenchymal and non-parenchymal sinusoidal cells given the conditioned medium from cultured CRC cells (HT-29 CRC cell line). This gene subset was also selected for further analysis.

TABLE 2

| | Downregulated Genes |
|---|---|
| ACTG1 | Actin, gamma 1 |
| ARPC2 | Actin related protein 2/3 complex, subunit 2, 34 kDa |
| BMP7 | Bone morphogenetic protein-7 |
| CALM1 | Calmodulin 1 (phosphorylase kinase, delta) |
| CAPG | Capping protein (actin filament), gelsolin-like |
| CEACAM1 | Carcinoembryonic antigen-related cell |
| | adhesion molecule 1 (biliary glycoprotein) |
| COL18A1 | Collagen, type xviii, alpha 1 |
| ERBB2IP | Erbb2 interacting protein |
| H2AFY | H2A histone family, member Y |
| H3F3B | H3 histone, family 3B (H3.3B) |
| HIST3H3 | Histone cluster 3, H3 |
| IFITM2 | Interferon induced transmembrane protein 2 (1-8D) |
| IGF1 | Insulin-like growth factor-1 |
| ING1 | Inhibitor of growth family, member 1 |
| NCL | Nucleolin |
| NGF | Nerve growth factor-beta |
| NOS2 | Nitric oxide synthase 2, inducible |
| PLP2 | Proteolipid protein 2 (colonic epithelium-enriched) |
| PPIA | Peptidylprolyl isomerase A (cyclophilin A) |
| RPIA | Ribose 5-phosphate isomerase A (ribose 5-phosphate |
| | epimerase) |
| RPL10 | Ribosomal protein L10 |
| RPS23 | Ribosomal protein S23 |
| RPS27 | Ribosomal protein S27 (metallopanstimulin 1) |
| RPS3A | Ribosomal protein S3A |
| SDC1 | Syndecam-1 |
| TMSB10 | Thymosin, beta 10 |
| TUBA4A | Tubulin, alpha 4a |
| YWHAB | Tyrosine 3-monooxygenase/tryptophan 5-monooxygenase |
| | activation protein, beta polypeptide |

Table 3 below shows liver prometastatic gene families (Inflammatory, Immune Regulation, Metabolic Bioprotection, and Fibrogenic Regeneration) of the thirty-one, two-fold upregulated and two-fold down-regulated genes of Tables 1 and 2 whose altered expression level in tumor-unaffected hepatic tissue is associated with liver metastasis growth in patients with CRC. The functional gene classification activity was performed manually by accessing the Gene Ontology and PubMed databases and is based on known biopathological functions assigned individually to studied genes. Below in Table 3 are listed and sorted by functional categories these 31 liver prometastatic genes.

TABLE 3
Liver Prometastatic Genes Sorted by Functional Categories

| Metabolic | Immune | | Fibrogenic |
|---|---|---|---|
| Bioprotection | Regulation | Inflammatory | Regenerative |
| Genes | Genes | Genes | Genes |
| TXN | KNG1 | ID-1 | RPS27 |
| PRDX4 | CEACAM-1 | IL-18 | RPS23 |
| NOS2 | BMP-7 | TNF-alpha | DDR2 |
| GAPDH | SDC-1 | VEGF-A | TGFB1 |
| MTE1 | COL18-A1 | EPHA-1 | VTN |
| HP | IL-10 | TNFSF14 | NGF |
| CRP | ICAM-1 | CYP2E1 | IGF-1 |
| ERBB21P | MRC1 | ADH1B | |

A first teaching use for the present invention concerns identifying metastasis-associated genes in the tumor-unaffected hepatic tissue of Stage-IV cancer patients with metastatic CRC. As discussed in connection with FIG. 1, comparative transcriptome profiling using RNA from hepatic CRC metastases, tumor-unaffected hepatic tissue, and peripheral mononuclear blood cells uncovered approximately 122 genes specifically over-expressed and approximately 28 genes specifically under-expressed, each group being more than two-fold overexpressed or under-expressed in tumor-unaffected hepatic tissue from Stage-IV cancer patients with metastatic CRC. These genes are identified in Tables 1 and 2 above. Upregulated and downregulated gene sets were selected for further analysis. Transcriptome profiling was obtained from surgically removed liver specimens and archived biopsies of patient tissue. Table 3 shows a subset of these liver-associated genes (over-expressed and under-expressed genes) isolated according to their association with cancer-related cellular functions of inflammation, immune regulation, metabolic bioprotection and regeneration, i.e., functional categories. Further laboratory tests were performed on this subset of liver-associated genes to categorize them according to additional prometastatic criteria including (1) altered expression level in tumor-unaffected hepatic tissue associated with liver metastasis growth in patients with CRC, (2) altered expression in cultured liver parenchymal and non-parenchymal cells exposed to soluble factors from cultured CRC cells, and (3) altered expression associated with experimental hepatic colonization and growth of circulating CRC cells in animal models of CRC.

Table 4 below shows actual clinical data taken from forty-five patients (29 patients with CRC and 16 without CRC) that were included in the study on the expression pattern of liver prometastatic genes in hepatic biopsies from patients with and without CRC where TNM indicates tumor node metastasis stage.

TABLE 4

| | Patients with CRC | | Patients without CRC | |
|---|---|---|---|---|
| Clinical Parameters | No. | % | No. | % |
| Gender | | | | |
| Female | 10 | 34 | 8 | 50 |
| Male | 19 | 66 | 8 | 50 |
| Average Age | 58 | — | 57 | — |
| Metabolic Syndrome | 14 | 48 | 7 | 43 |
| Cholelithiasis | 0 | 0 | 16 | 100 |
| TNM stage | | | | |
| I | 0 | 0 | 0 | 0 |
| II | 0 | 0 | 0 | 0 |
| III | 12 | 42 | 0 | 0 |
| IV | 17 | 58 | 0 | 0 |
| Tumor localization | | | | |
| Left-sided Colon | 11 | 39 | 0 | 0 |
| Right-sided Colon | 10 | 34 | 0 | 0 |
| Rectum | 6 | 20 | 0 | 0 |
| Others (gastric, duodenum) | 2 | 7 | 0 | 0 |

Table 5 below shows measurement data indicative of the thirty-one two-fold plus upregulated and down-regulated liver prometastatic gene expression levels under investigation in patients with and without CRC. The data shown therein represents average normalized (Ct ratio of studied gene/Ct of constitutive gene) Ct (cycle threshold) values ±SD (standard deviation) as well as mean probability values “p-values.”

TABLE 5

| | | | Average | [symbol] -Student [symbol] -Mann [symbol] Whitney [symbol] | |
|---|---|---|---|---|---|
| | | | Ct [symbol] Norm. [symbol] | [symbol] (p [symbol] Value) | [symbol] (p [symbol] Value) [symbol] |
| | | Without [symbol] CR [symbol] [symbol] | 0.812 | | |
| [symbol] | TXN | With [symbol] CR [symbol] [symbol] | 0.856 | 0.549 | 0.436 |
| | | Without [symbol] CR [symbol] [symbol] | 0.852 | | |
| [symbol] | PRDX4 | With [symbol] CR [symbol] [symbol] | 0.878 | | 0.086 |
| | | Without [symbol] CR [symbol] [symbol] | 0.892 | | |
| [symbol] | MT1E | With [symbol] CR [symbol] [symbol] | 0.970 | 0.026 | 0.014 |
| | | Without [symbol] CR [symbol] [symbol] | 1.000 | | |
| [symbol] | ERBB2IP | With [symbol] CR [symbol] [symbol] | 0.965 | 0.010 | 0.010 |
| | | Without [symbol] CR [symbol] [symbol] | 0.949 | | |
| [symbol] | NOS2 | With [symbol] CR [symbol] [symbol] | 1.175 | | 0.084 |
| | | Without [symbol] CR [symbol] [symbol] | 1.193 | | |
| [symbol] | HP | With [symbol] CR [symbol] [symbol] | 0.624 | 0.775 | 0.335 |
| | | Without [symbol] CR [symbol] [symbol] | 0.627 | | |
| [symbol] | CRP | With [symbol] CR [symbol] [symbol] | 0.833 | 0.004 | 0.002 |
| | | Without [symbol] CR [symbol] [symbol] | 0.908 | | |
| [symbol] | BMP7 | With [symbol] CR [symbol] [symbol] | 1.327 | 0.514 | 0.348 |
| | | Without [symbol] CR [symbol] [symbol] | 1.309 | | |
| [symbol] | SDC1 | With [symbol] CR [symbol] [symbol] | 0.921 | | 0.045 |
| | | Without [symbol] CR [symbol] [symbol] | 0.893 | | |
| [symbol] | IGF1 | With [symbol] CR [symbol] [symbol] | 0.975 | 0.593 | 0.741 |
| | | Without [symbol] CR [symbol] [symbol] | 0.970 | | |
| [symbol] | COL18A1 | With [symbol] CR [symbol] [symbol] | 0.842 | | 0.013 |
| | | Without [symbol] CR [symbol] [symbol] | 0.821 | | |
| [symbol] | ICAM1 | With [symbol] CR [symbol] [symbol] | 0.965 | | 0.000 |
| | | Without [symbol] CR [symbol] [symbol] | 0.019 | | |
| [symbol] | KNG1 | With [symbol] CR [symbol] [symbol] | 0.794 | | 0.042 |
| | | Without [symbol] CR [symbol] [symbol] | 0.776 | | |
| [symbol] | IL10 | With [symbol] CR [symbol] [symbol] | 1.081 | 0.000 | 0.000 |
| | | Without [symbol] CR [symbol] [symbol] | 1.130 | | |
| [symbol] | CEACAM1 | With [symbol] CR [symbol] [symbol] | 0.963 | 0.878 | 0.864 |
| | | | 0.962 | | |
| [symbol] | MRC1 | Without [symbol] CR [symbol] [symbol] | 0.976 | | 0.000 |
| | | With [symbol] CR [symbol] [symbol] | 1.018 | | |
| [symbol] | EPHA1 | Without [symbol] CR [symbol] [symbol] | 1.032 | 0.002 | 0.002 |
| | | With [symbol] CR [symbol] [symbol] | 1.002 | | |
| [symbol] | TNFSF14 | Without [symbol] CR [symbol] [symbol] | 0.988 | | 0.025 |
| | | With [symbol] CR [symbol] [symbol] | 1.018 | | |
| [symbol] | CYP2E1 | Without [symbol] CR [symbol] [symbol] | 0.717 | 0.000 | 0.000 |
| | | With [symbol] CR [symbol] [symbol] | 0.688 | | |
| [symbol] | ADH1B | Without [symbol] CR [symbol] [symbol] | 0.734 | 0.001 | 0.001 |
| | | With [symbol] CR [symbol] [symbol] | 0.698 | | |
| [symbol] | ID1 | Without [symbol] CR [symbol] [symbol] | 0.908 | 0.007 | 0.001 |
| | | With [symbol] CR [symbol] [symbol] | 0.935 | | |
| [symbol] | TNF | Without [symbol] CR [symbol] [symbol] | 1.060 | 0.000 | 0.000 |
| | | With [symbol] CR [symbol] [symbol] | 1.164 | | |
| [symbol] | IL18 | Without [symbol] CR [symbol] [symbol] | 1.052 | 0.000 | 0.000 |
| | | With [symbol] CR [symbol] [symbol] | 1.114 | | |
| [symbol] | VEGFA | Without [symbol] CR [symbol] [symbol] | 0.910 | 0.882 | 0.792 |
| | | With [symbol] CR [symbol] [symbol] | 0.911 | | |
| [symbol] | RPL23 | Without [symbol] CR [symbol] [symbol] | 0.807 | 0.766 | 0.712 |
| | | With [symbol] CR [symbol] [symbol] | 0.808 | | |
| [symbol] | RPS27 | Without [symbol] CR [symbol] [symbol] | 0.763 | 0.148 | 0.178 |
| | | With [symbol] CR [symbol] [symbol] | 0.755 | | |
| [symbol] | VTN | Without [symbol] CR [symbol] [symbol] | 0.707 | | 0.428 |
| | | With [symbol] CR [symbol] [symbol] | 0.702 | | |
| [symbol] | NGF | Without [symbol] CR [symbol] [symbol] | 1.141 | 0.019 | 0.007 |
| | | With [symbol] CR [symbol] [symbol] | 1.113 | | |
| [symbol] | TGFB1 | Without [symbol] CR [symbol] [symbol] | 0.972 | 0.934 | 0.989 |
| | | With [symbol] CR [symbol] [symbol] | 0.973 | | |
| [symbol] | DDR2 | Without [symbol] CR [symbol] [symbol] | 1.045 | 0.438 | 0.421 |
| | | | 1.038 | | |

[symbol]

indicates data missing or illegible when filed

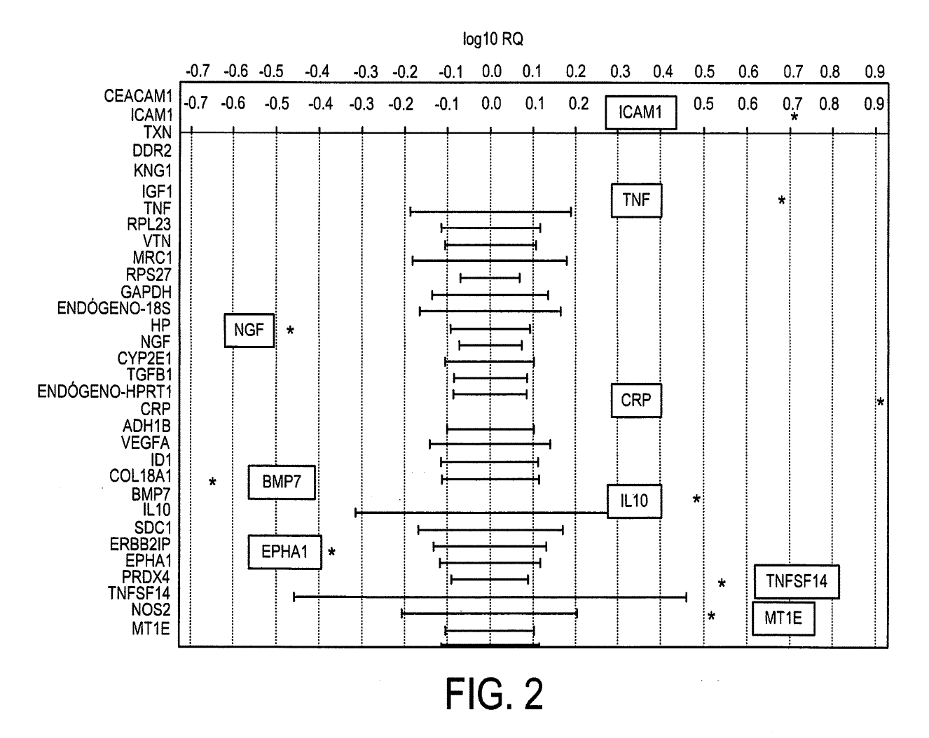

FIG. 2 depicts a logarithmic scale representation of the relative quantification (RQ) values of liver prometastatic gene expressions in CRC patients with respect to same values in patients without CRC. Error bars indicate maximum and minimum RQ values. “*” indicates statistically significant values where probability p<0.05 (i.e., five percent).

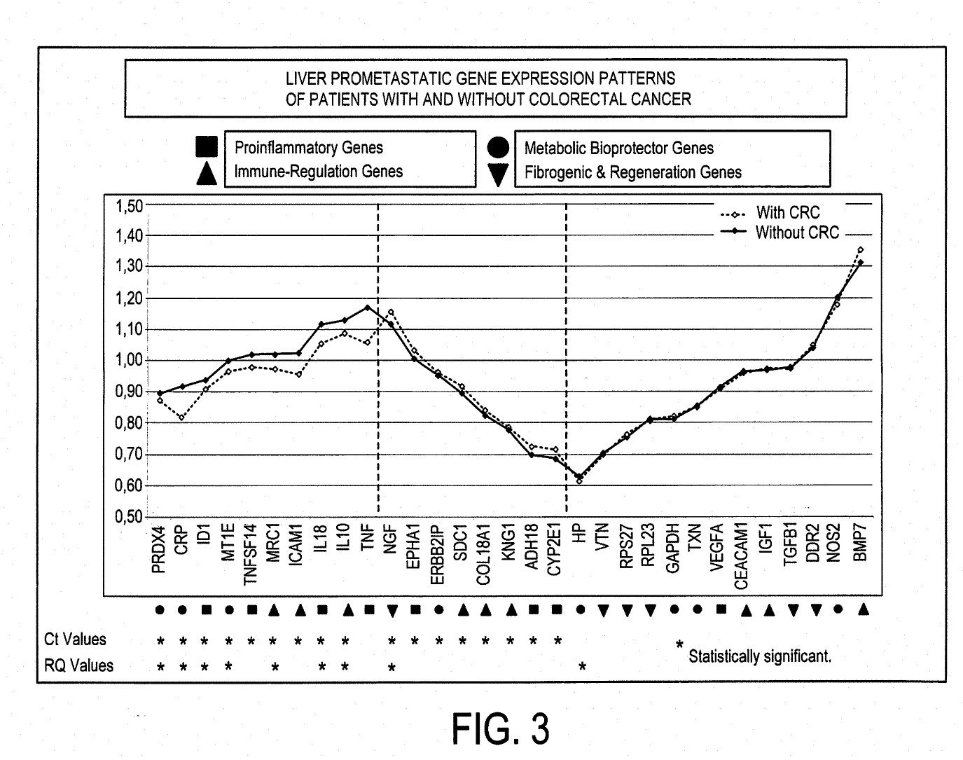

FIG. 3 depicts liver prometastatic gene expression patterns (color-coded in provisional application according to functional category but here, ▪ indicates proinflammatory genes, ▴ indicates immune-regulation genes, ● indicates metabolic bioprotection genes, and ▾ indicates fibrogenic and regeneration genes) in patients with and without CRC. Differences between patients with and without CRC were statistically significant according to a U-Man Whitney test (p<0.05). Statistically significant genes are identified by “*” denoted in the lower legend of FIG. 3. Advantageously, by examining the expression levels of one or more statistically significant genes in group 1 and/or group 2 of patients with CRC relative to a control or reference (i.e., gene expression levels of persons free of CRC), rather than waiting for clinical signs to become apparent by imaging or non-biochemical changes in the microenvironment, one may detect metastatic cancer in the hepatic biochemical microenvironment to enable very early treatment and potential eradication of metastatic cancer cells.

An aspect of the invention includes a complementary diagnostic test to detect “liver prometastatic reaction level and class” in patients with CRC without metastatic disease. Expression of liver prometastatic genes in hepatic tissue selected above was next studied in twenty-nine patients with CRC (at stages III and IV) and sixteen patients without CRC used as controls. Table 4 details clinical information about the patients involved in the study. Based on normalized Ct values (i.e., cycle counts during the PCR process), Table 5 shows average values of the gene expression levels for the 31 genes involved for the 29 patients with CRC. As reflected in FIG. 3, expression levels were significantly (probability p<0.05) increased for ten liver prometastatic genes (group 1) and decreased in eight genes (group 2), with non-statistically significant (i.e., insignificant) changes in twelve to thirteen genes, when comparing patients with (29 patients) and without (16 patients) CRC. The vertical axis of FIG. 3 reflects Cₜ data relative to a control (i.e., reference values), or ratio of the Cₜ cycle count (i.e., Ct value) of the sample under examination relative to the Cₜ cycle count of a control or reference Cₜ value. Regarding group 1 genes of patients with CRC, i.e., the dotted trace, detection of an expression level signal at a lower cycle count Cₜ in the PCR process indicates a higher gene expression level. Detection of an expression level signal for each of the group 2 genes of patients with CRC occurred at a higher cycle count Cₜ as reflected in the middle portion of the dotted trace of FIG. 3. However, no statistically significant changes in gene expression levels of the latter group of twelve to thirteen genes (group 3) were detected when comparing CRC patients with and without hepatic metastases (or between CRC patients and CRC-free patients as controls) suggesting that detected liver prometastatic gene expression changes in tumor unaffected hepatic tissue nevertheless occurred in the liver of CRC patients irrespective of having or not having metastases. In addition, correlation of respective gene expression levels of group 3 genes was deemed required to validate the efficacy of the relative expression levels of group 1 and group 2 genes. In other words, without congruence of the expression levels of patients with and without CRC among group 3 genes, the conditions denoted for group 1 and group 2 genes would not be valid. According to an aspect of the present invention, an output device of a rule-based system with the aid of programming instructions may produce a plot of Ct values of genes denoted in FIG. 3, either on a graphic display device or a printed chart, in order to guide medical professional in advising patients about metastatic cancer. To produce a result, the rule-based system or device may give weight to the respective gene expression levels and/or provide selection of only certain ones of the genes deemed significant to take into consideration.

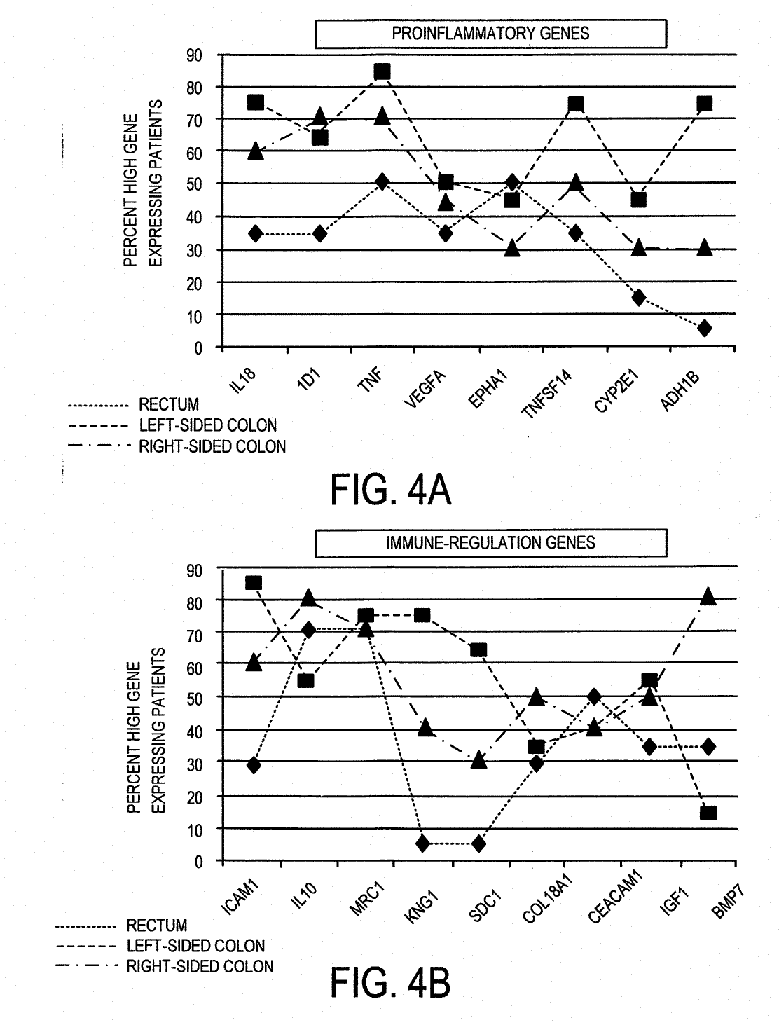

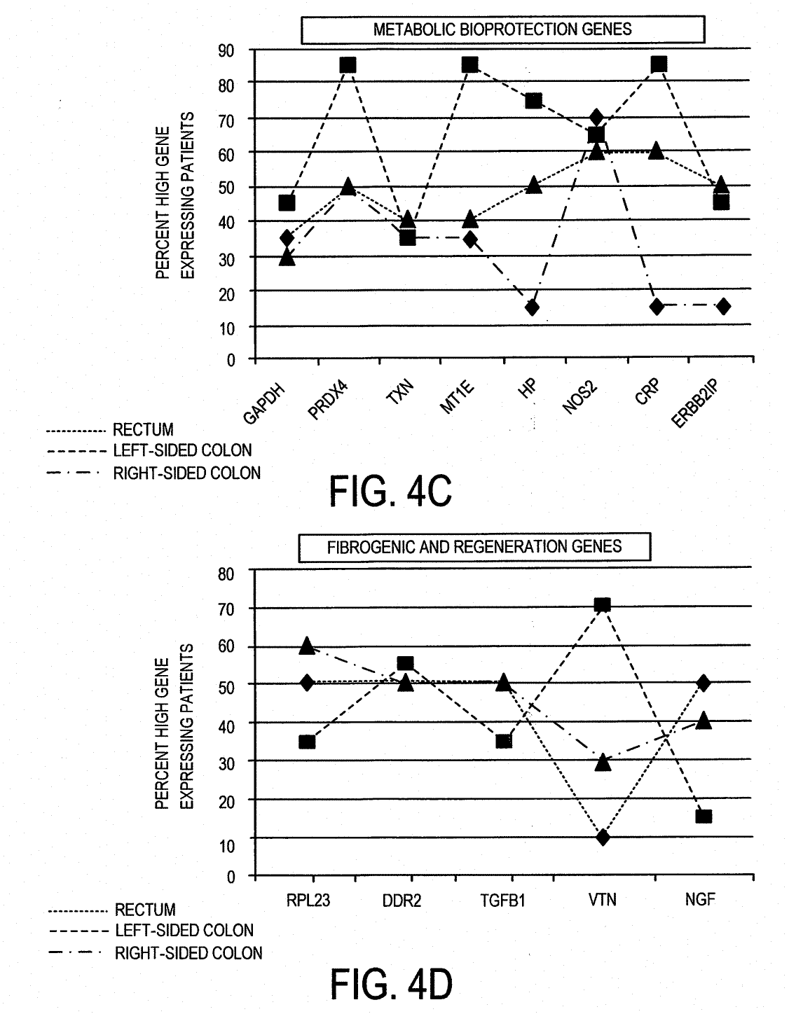

FIGS. 4A-4D and Table 6 further indicate that expression levels of the various genes differ according to the anatomical location of the patient's primary CRC, i.e., in the rectum, left-side colon or the right-side colon. Therefore, a liver prometastatic reaction occurs in the liver of patients with CRC prior to metastasis development and, in accordance with another aspect of the invention, by scoring number and intensity of gene changes according to the relationships shown in FIGS. 4A-4D, the inventive system or device may provide an indication of the genesis of prometastatic hepatic cancer.

It is also noted that the majority of proinflammatory (seven out of eight) and immune regulation (six out of nine) liver prometastatic genes, but only a minority of fibro-regenerative (one out of five) and metabolic bio-protective (three out genes eight) were significantly (p<0.05) changed in patients with CRC versus patients without CRC (Table 3, Table 6, and FIGS. 4A-4D). This suggests that in addition to the number of changed genes, the kind of changed genes in functional terms defines the Liver Prometastatic Reaction Class in the liver of patients with CRC. Both the number and functional categories of liver prometastatic genes changed in patients with CRC may serve as a complementary diagnostic test for the quantitative assessment of liver metastasis risk and recurrence in patients with CRC, and thus, as a precursor, may form the basis of a method of detecting occult CRC subclinically in patients having no clinical symptoms of CRC at all. A processing system to receive data inputs and appropriate program instructions may be utilized to automatically output this determination on a display device or other output.

TABLE 6
Liver prometastatic gene expression level by functional
category and anatomic location of the primary CR

[symbol]

| | Gene | | | |
|---|---|---|---|---|
| | Expres- | | LEFT- | RIGHT- |
| Liver Prometastatic | sion | | SIDED | SIDED |
| Genes | Level | RECTUM | COLON | COLON |
| PRO- | HIGH | — | IL18 | ID1 |
| INFLAMMATORY | | | ID1 | TNF |
| GENES | | | TNF | |
| | | | TNFSF14 | |
| | | | ADH1B | |
| | LOW | IL18 | — | ADH1 [symbol] |
| | | ID1 | | CYP2E1 |
| | | VEGFA | | |
| | | TNFSF14 | | |
| | | ADH1B | | |
| | | CYP2E1 | | |
| IMMUNO | HIGH | IL10 | ICAM1 | [symbol] 10 |
| REGULATION | | MRC1 | MRC1 | MRC1 |
| GENES | | | KNG1 | EMP7 |
| | | | [symbol] DC1 | |
| | LOW | ICAM1 | [symbol] MP7 | KNG1 |
| | | KNG1 | | SOC1 |
| | | SDC1 | | |
| | | IGF1 | | |
| | | BMP7 | | |
| METABOLIC | HIGH | NO52 | PRXD4 | — |
| BIOPROTECTION | | | MTE1 | |
| GENES | | | HP | |
| | | | NO [symbol] 2 | |
| | | | CRP | |
| | LOW | GAPDH | TXN | — |
| | | TXN | | |
| | | MTE1 | | |
| | | HP | | |
| | | CRP [symbol] R332IP | | |
| [symbol] OGENIC AND | HIGH | — | VTN | — |
| REGENERATION | LOW | VTN | RLP23 | VTN |
| GENES | | | TGFB1 | NGF |
| | | | NGF | |

[symbol]

indicates data missing or illegible when filed

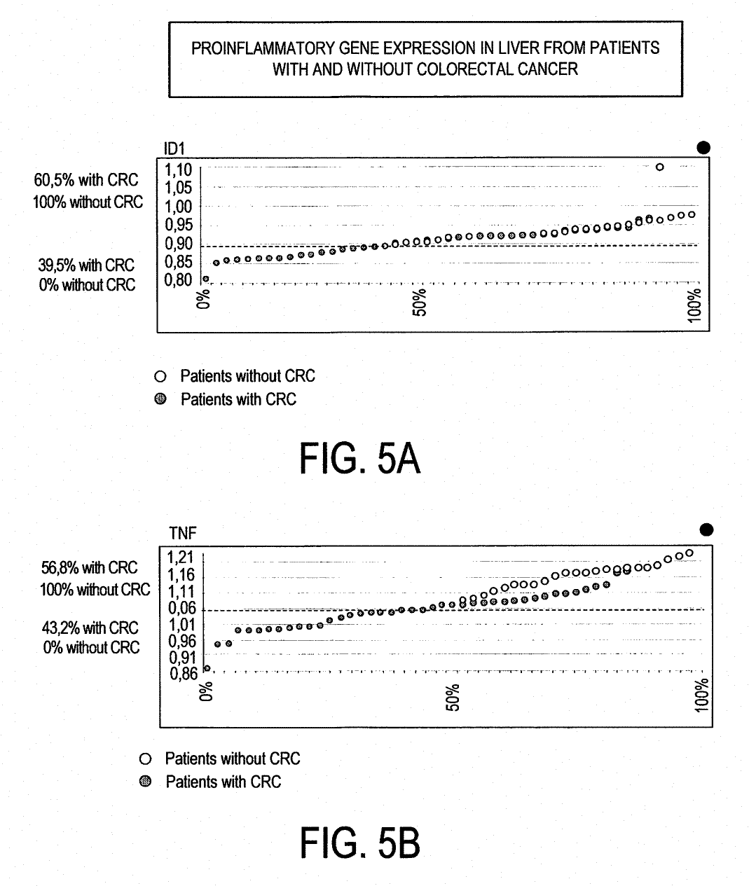

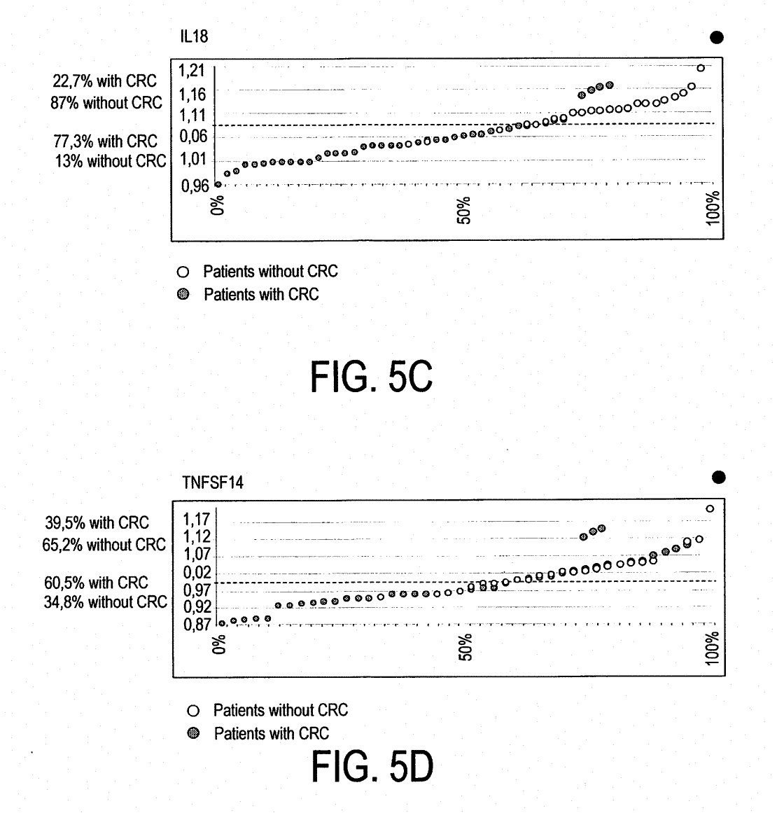

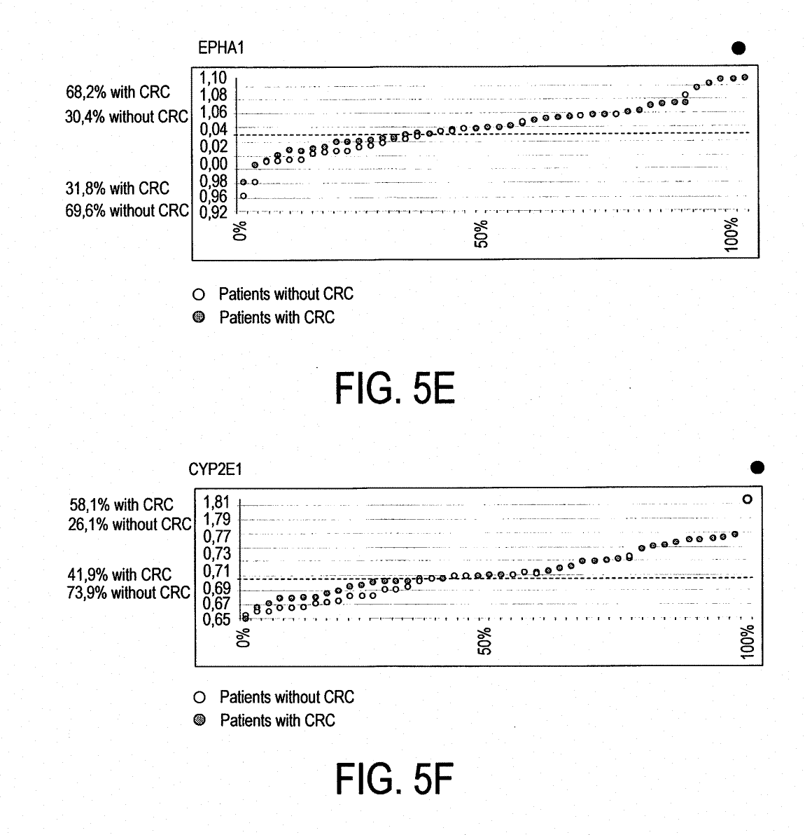

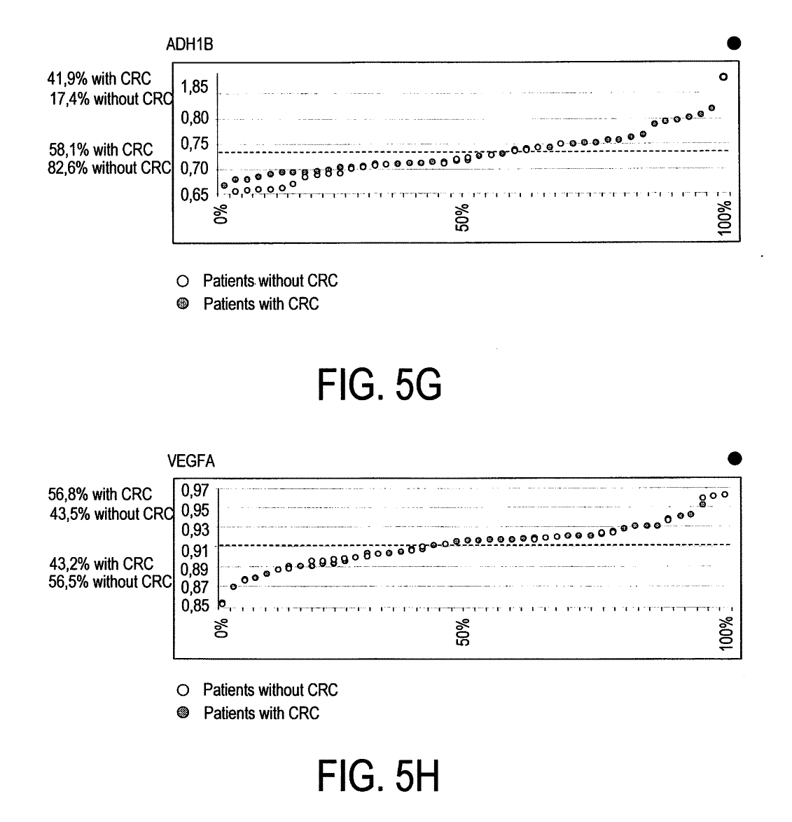

FIG. 5 shows proinflammatory gene expressions in liver from patients with and without CRC. Data are represented as increasing distribution of mean values. Data express normalized Ct values (Ct Ratio of studied gene/Ct of constitutive gene). The discontinuous line marks the intermediate point between the minimum and maximum ratios obtained for each gene (Y axis) for total number of analyzed samples (X axis) from patients with and without CCR.

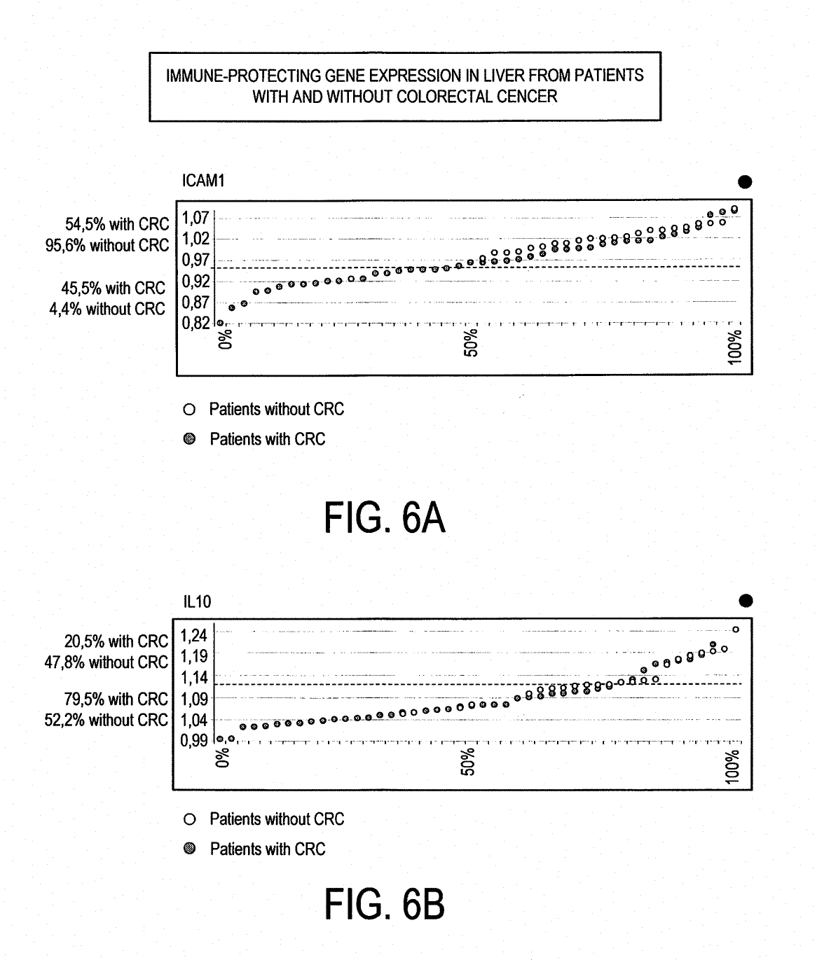

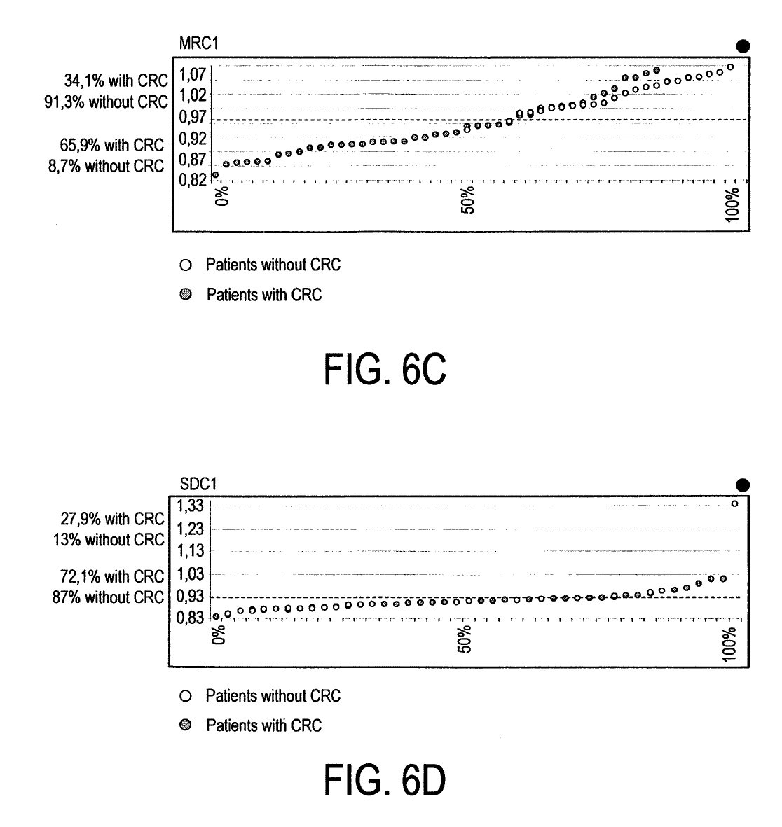

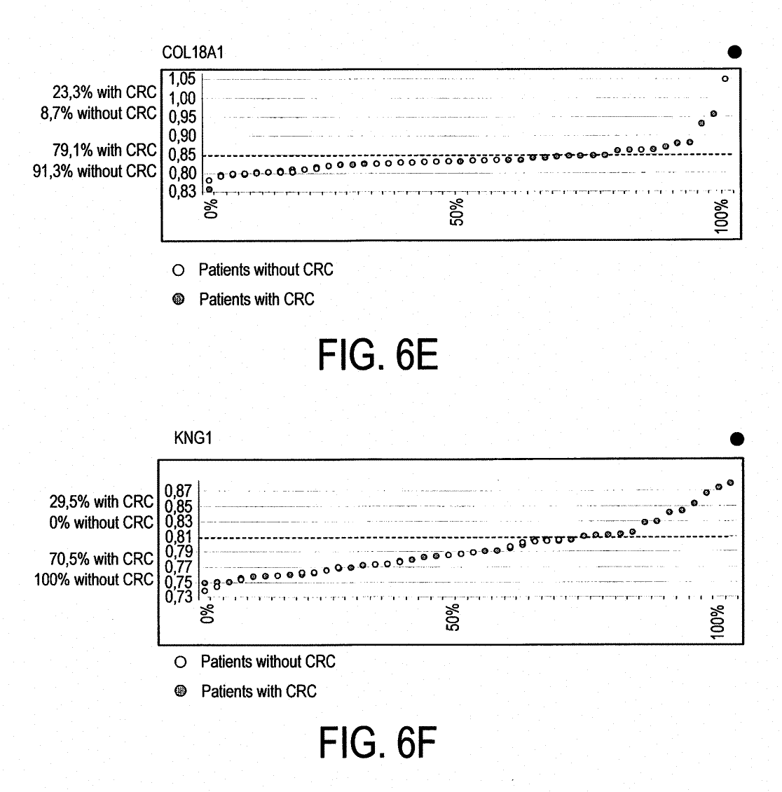

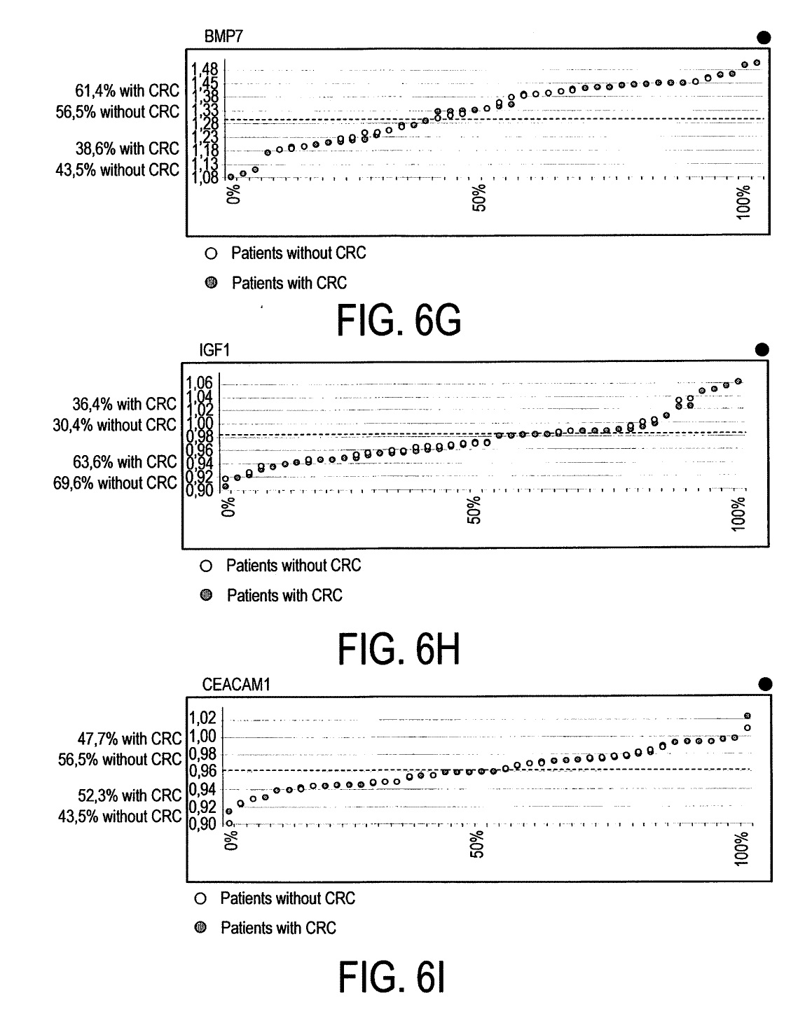

FIG. 6 depicts immuno-regulation gene expressions in liver from patients with and without CRC. Data are represented as increasing distribution of mean values. Data express normalized Ct values (Ct Ratio of studied gene/Ct of constitutive gene). The discontinuous line marks the intermediate point between the minimum and maximum ratios obtained for each gene (Y axis) for total number of analyzed samples (X axis) from patients with and without CCR.

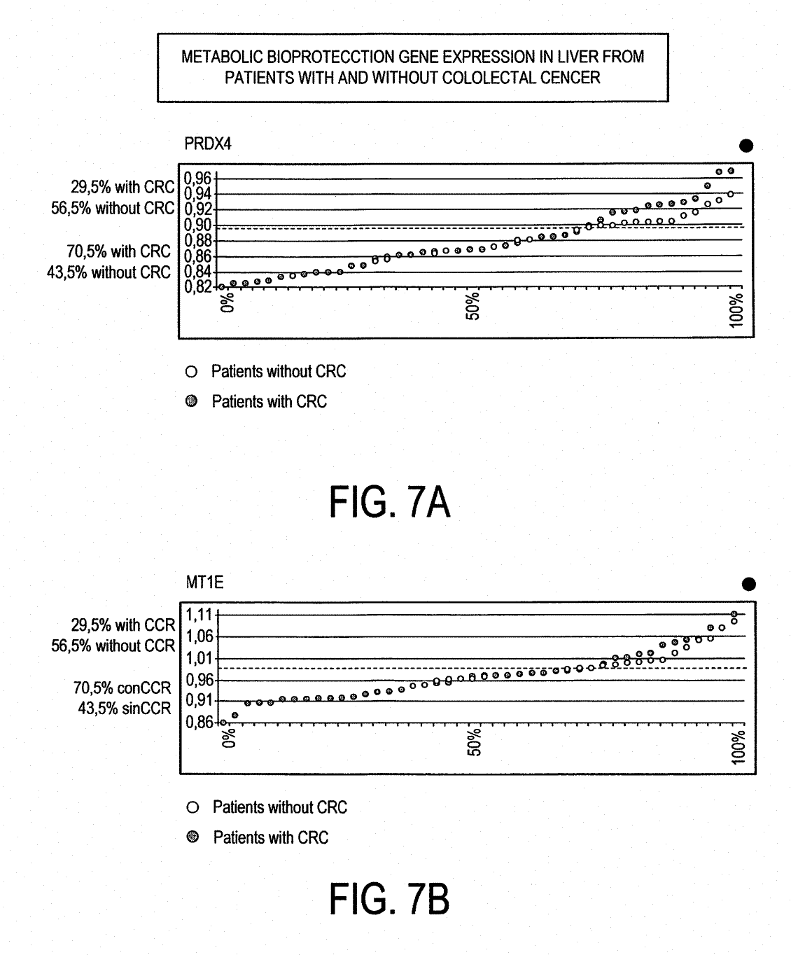

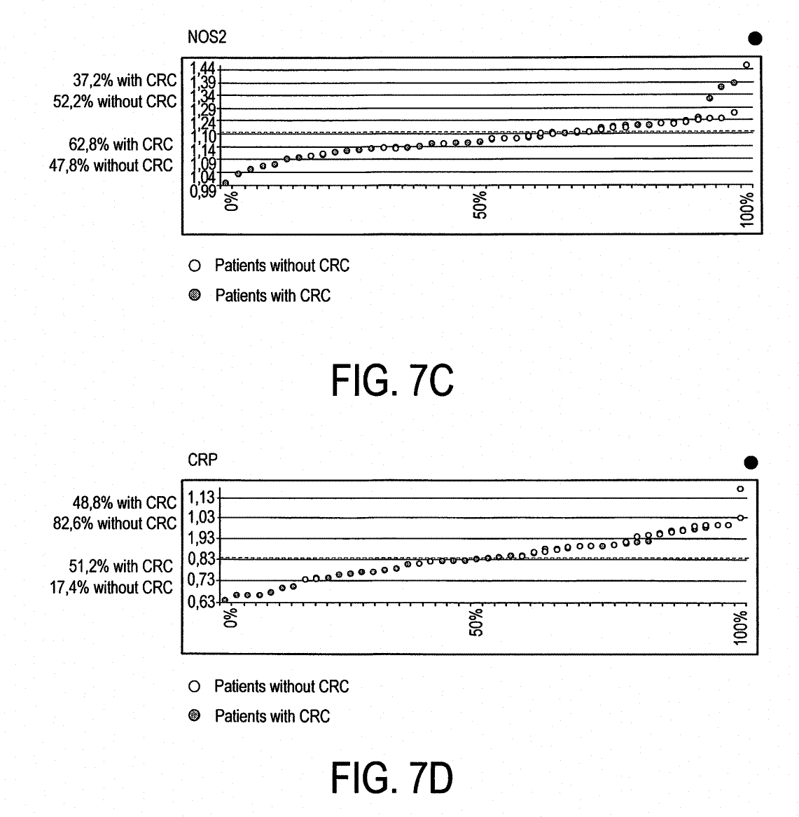

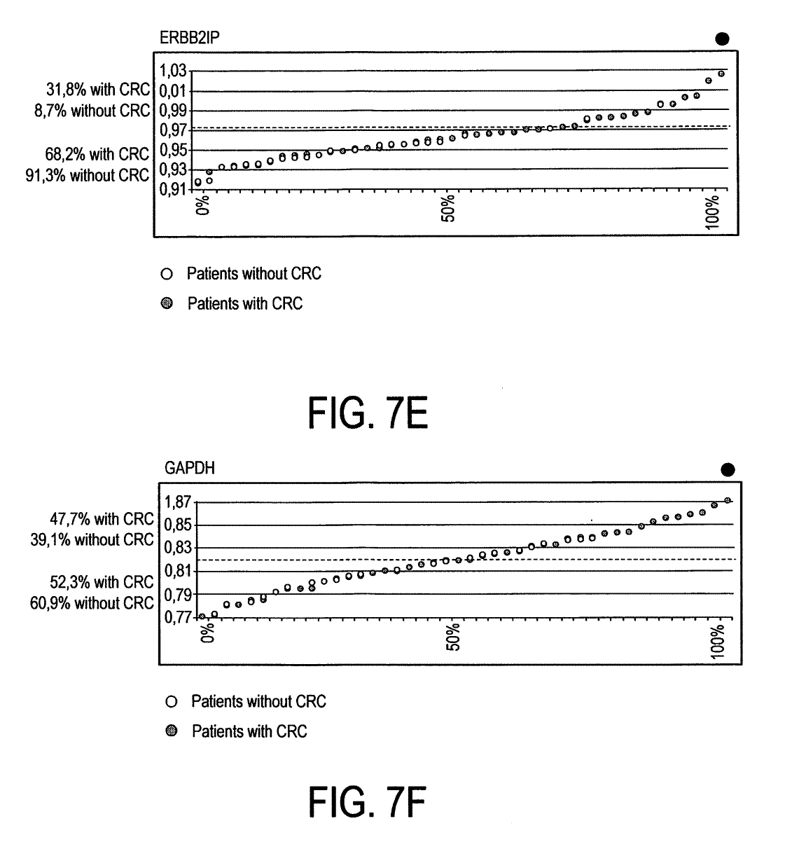

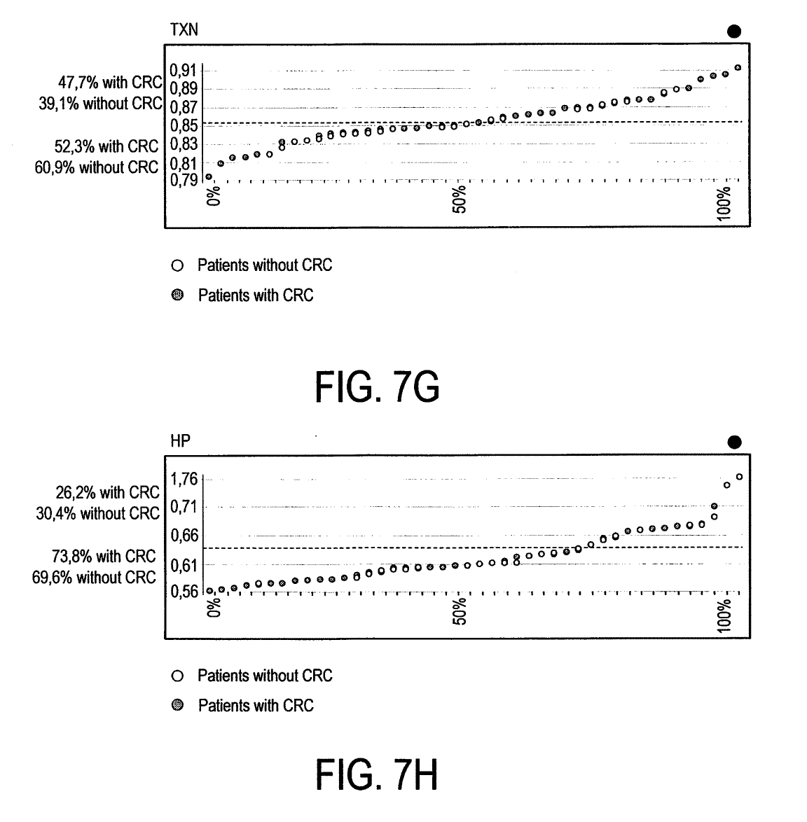

FIG. 7 depicts metabolic bioprotection gene expressions in liver from patients with and without CRC. Data are represented as increasing distribution of mean values. Data express normalized Ct values (Ct Ratio of studied gene/Ct of constitutive gene). The discontinuous line marks the intermediate point between the minimum and maximum ratios obtained for each gene (Y axis) for total number of analyzed samples (X axis) from patients with and without CRC.

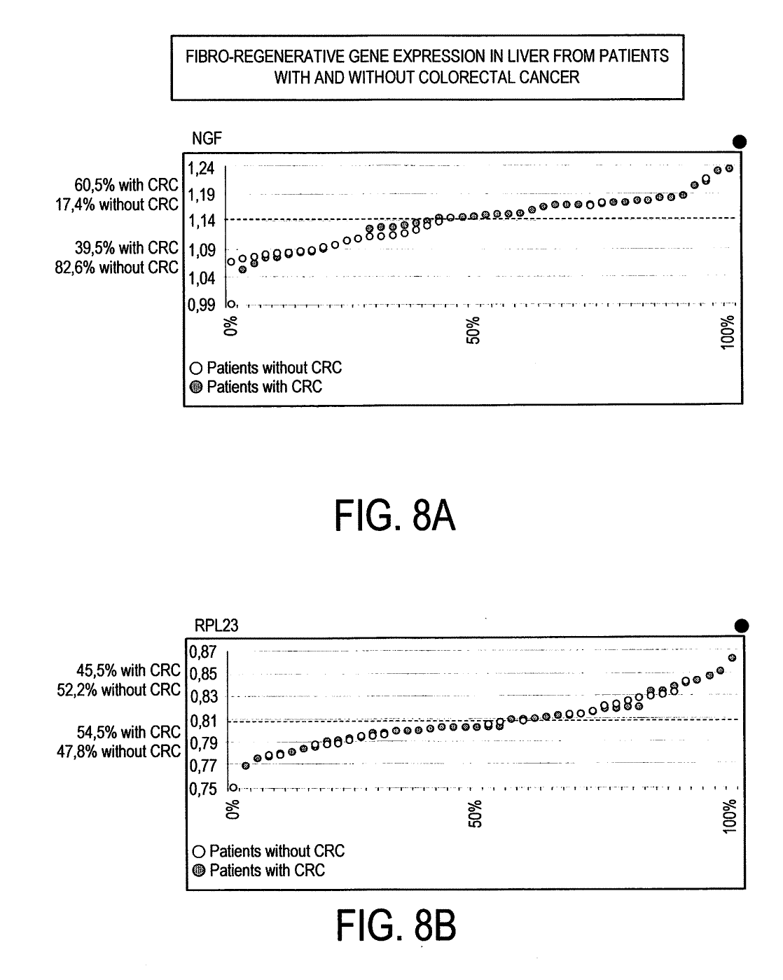

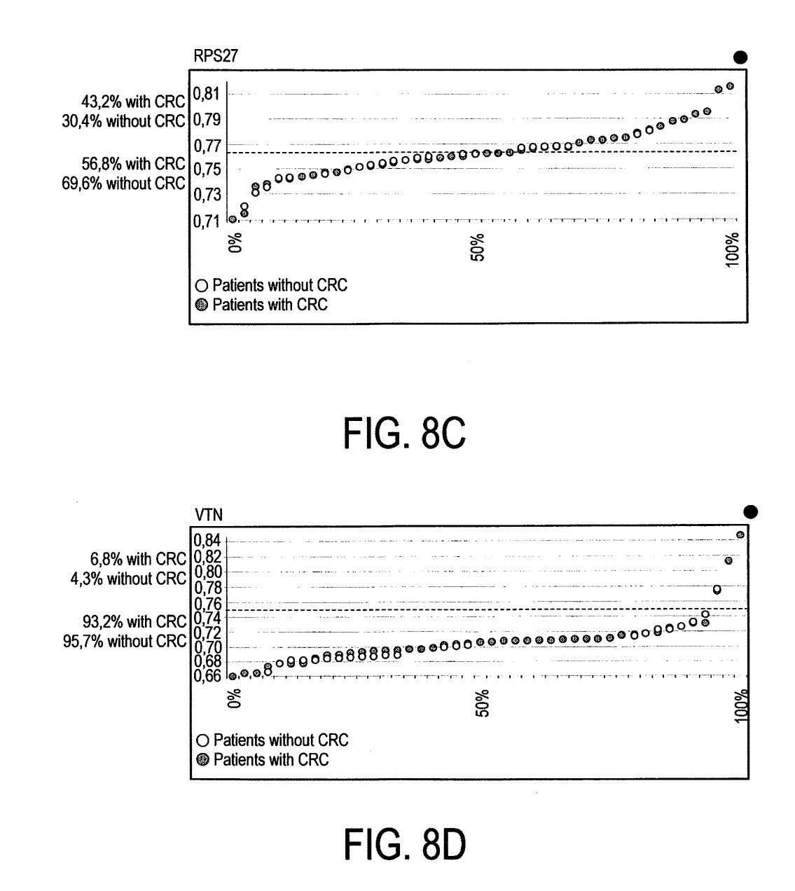

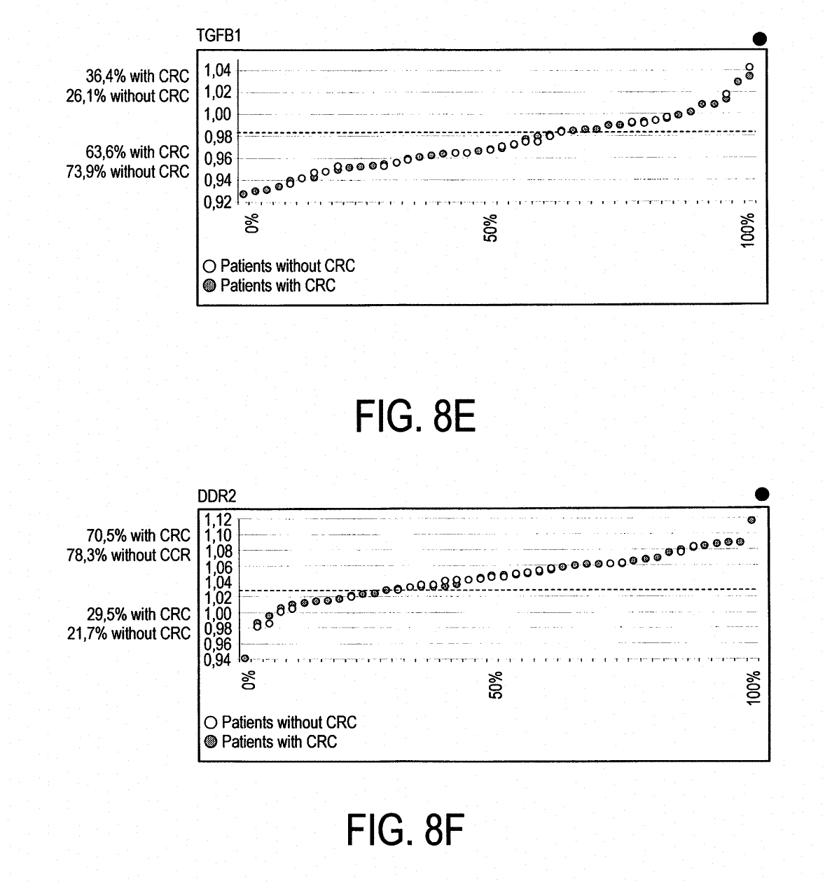

FIG. 8 depicts fibrogenic and regeneration gene expressions in liver from patients with and without CRC. Data are represented as increasing distribution of mean values. Data express normalized Ct values (Ct Ratio of studied gene/Ct of constitutive gene). The discontinuous line marks the intermediate point between the minimum and maximum ratios obtained for each gene (Y axis) for total number of analyzed samples (X axis) from patients with and without CRC.

Based on analyses illustrated in FIGS. 5-8, another aspect of the invention includes the device or apparatus performing an analysis of expression level data to provide a complementary diagnostic test to provide an alert of possible occult CRC in patients without clinical evidence of CRC but with other digestive system diseases that increase CRC risk, such as but not limited to cholelithiasis and metabolic syndrome. Comparative distribution of gene expression levels of selected genes of studied CRC patients and their controls without CRC, by their expression of liver prometastatic genes (as indicated by the analyses shown in FIGS. 5-8) demonstrate that those genes best contributing to the segregation of patients with and without CRC are Metabolic bioprotection genes PRDX4, MT1E, CRP and NOS2; Immune regulation genes ICAM1, IL10 and MRC1; and Proinflammatory genes ID1, TNF-a, IL18 and TNFSF14. All of these genes remarkably increased their expression levels in patients with CRC while decreased their expression levels in patients without CRC. On the contrary, immune-regulation genes SDC1, COL18A1 and KNG1, Proinflammatory genes EPHA1, CYP2E1, ADH1B, and fibrogenic/regeneration gene NGF increased their expression levels in patients without CRC while decreased their expression levels in patients with CRC, as indicated in FIGS. 5-8.

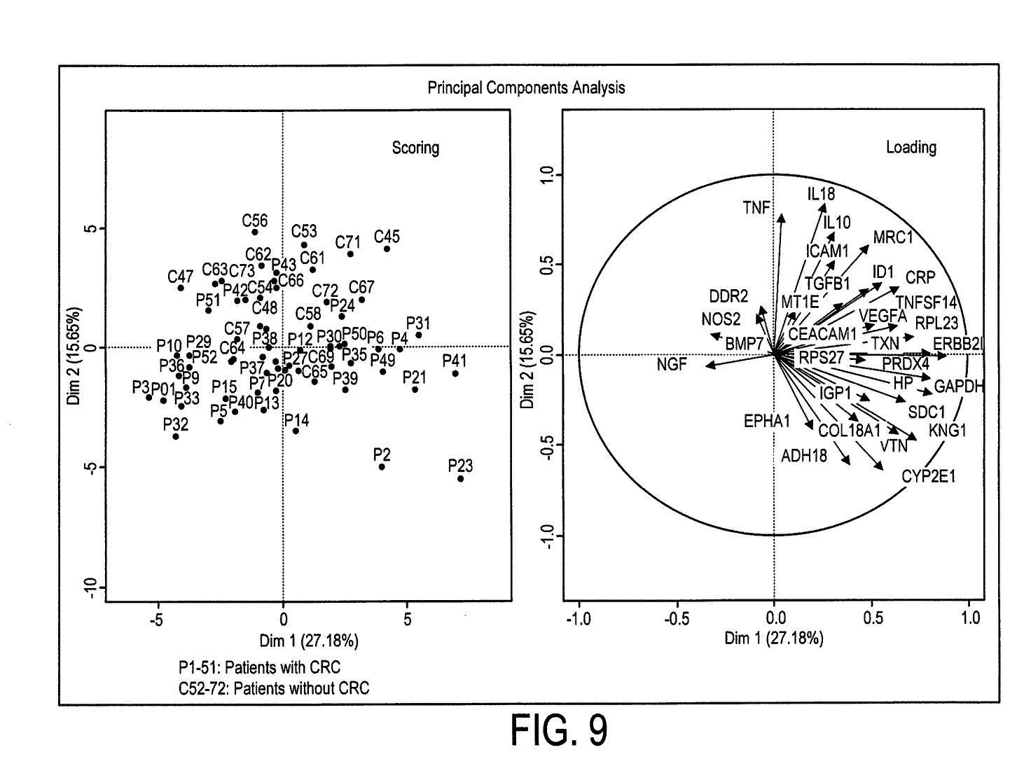

FIG. 9 shows a comparison of overall expression profiles across samples from patients with and without CRC in respective Scoring Value and Loading value charts, which also may be generated and analyzed by the invention device or apparatus. Scoring chart of FIG. 9 shows results for a Principal Component Analyses (PCA) of the data, which is used to emphasize variations and reveal data patterns of gene expressions in patients with (P1-P51) and without (C52-C72) CRC. The first principal component (Dimension 1) sets forth a 27.18% variation, whereas the second Dimension 2 sets forth a 15.65% variation. It is seen that Dimension 2 separated most of patients with CRC from patients without CRC.

A principal component analysis (PCA), multivariate regression analysis used to distinguish samples with multiple measurements was conducted, the results of which are shown in FIG. 9. Supervised discriminant analysis showed that liver prometastatic Immune regulation and proinflammatory genes were the most discriminative for patients with and without CRC.

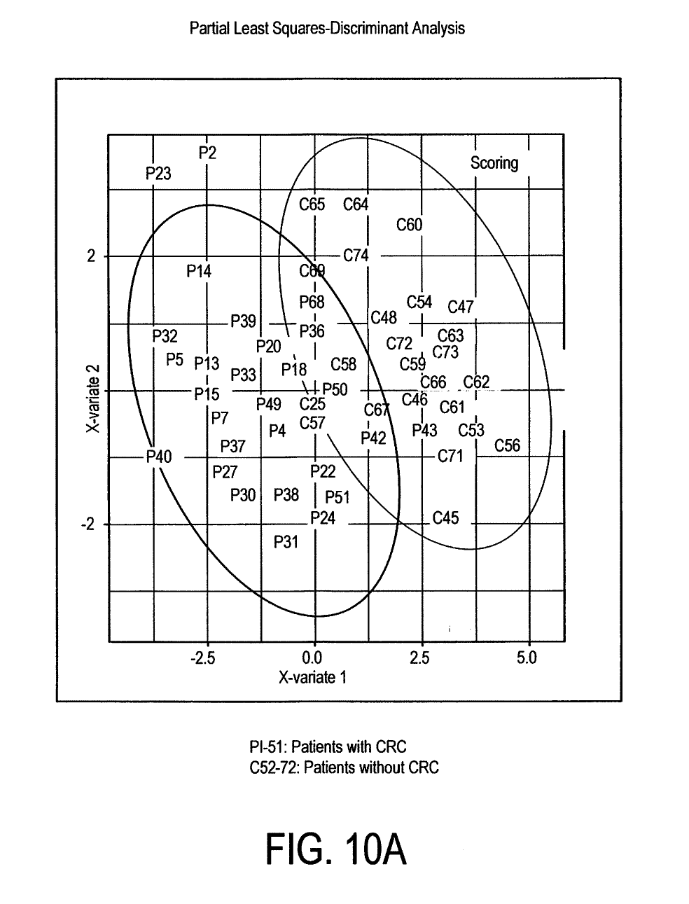

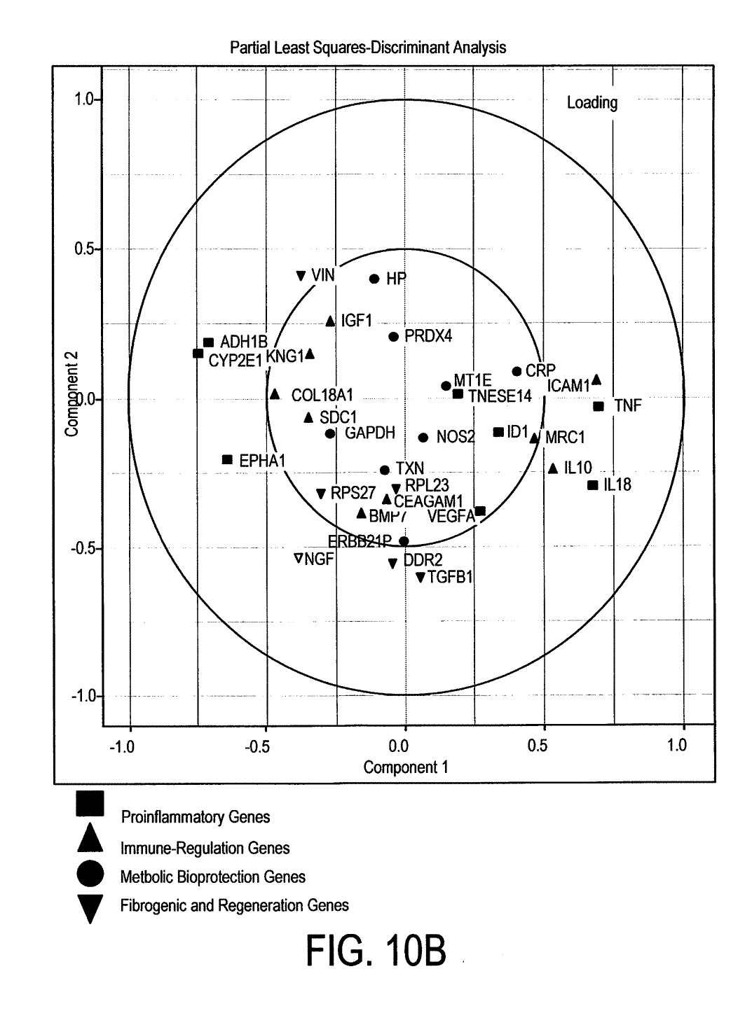

FIGS. 10A and 10B show a Partial Least Squares-Discriminant Analysis (PLS-DA) of gene expression data in respective Scoring and Loading plots, which are intended to discriminate between patients with and without CRC based on their hepatic expression levels of liver prometastatic genes. FIG. 10A depicts elliptical shapes adopted by lines that define the position coordinates of included patients (patients C52-72 without CRC and patients P1-51 with CRC). In this case, the discriminant capacity was associated with the first component in the analysis. FIG. 10B depicts position coordinates of liver prometastatic genes plotted in correlation circles whose diameters define influence of the genes in the prediction of the class of patient. In this case, metabolic bioprotection and fibrogenic/regeneration genes are in the smaller circle, indicating that their expression levels had less ability to predict the patient's class than Immune regulation and proinflammatory genes mainly located in the large correlation circle, which indicates a greater predictive capacity of the patient class. The inventive device or apparatus may also perform these analyses and provide a responsive output for use by a health care professional.

FIGS. 10A and 10B show results of a supervised discriminant analysis (i.e., a Partial

Least Squares-Discriminant Analysis, PLS-DA) to classify genes and patients by their correlation and ability to predict patients with and without CRC. The elliptical shapes adopted by lines in FIG. 10A define position coordinates of included patients and show that the discriminant capacity was associated with the first component in the analysis. Next, position coordinates of studied liver prometastatic genes were plotted in correlation circles, whose diameters define the influence of the genes in the prediction of the class of patient (FIG. 10B). Studied genes were distributed in correlation circles according to their functional category and once again, metabolic bioprotection and fibrogenic/regeneration genes were in the smaller circle, indicating that their expression levels had less ability to predict the patient's class, while Immune regulation and proinflammatory genes were mainly located in the large correlation circle, indicating their greater predictive capacity of the patient class.

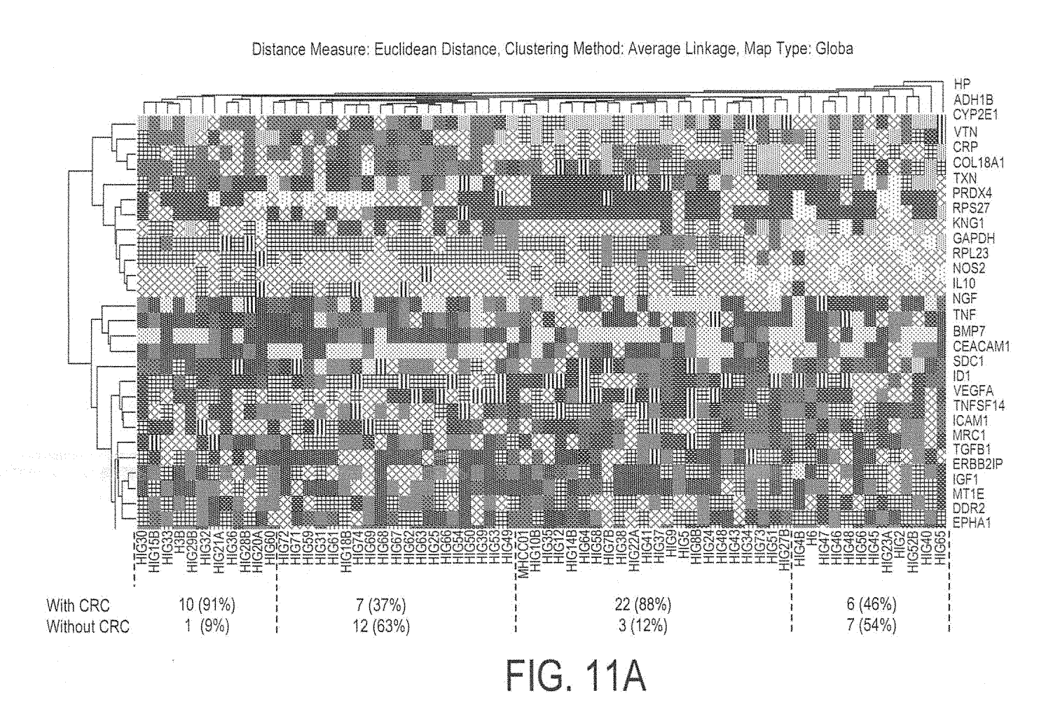

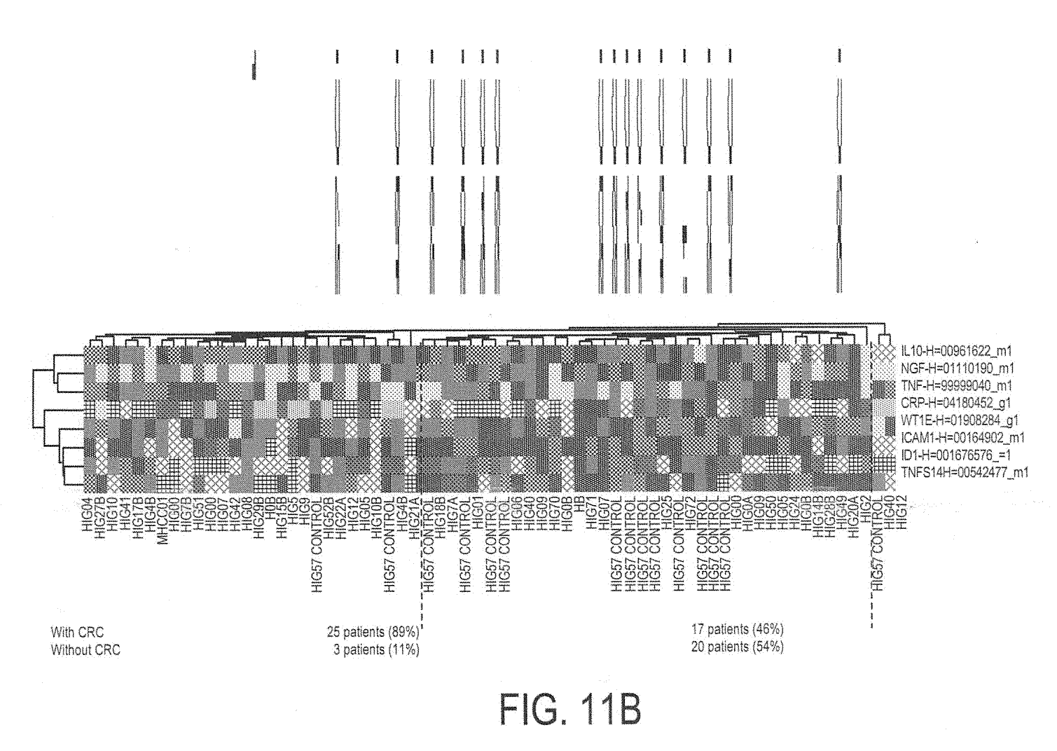

FIGS. 11A and 11B shows heatmaps for clustering patients with and without CRC according their liver prometastatic gene expression patterns based on ΔΔCt ratio. FIG. 11A shows four subgroups of patients with distinct gene expression patterns, two of them being enriched by patients with CRC and the two others by patients without CRC. FIG. 10B shows two subgroups with distinct gene expression patterns (genes with significant averages difference and significant RQ (relative quantification), enriched by either patients with or without CRC that were generated using the most discriminating genes. Some patients (noted in color green in the first and third groups) without CRC are seen to be grouped with patients with CRC suggesting they may have occult CRC (which was later confirmed by colonoscopy), while other patients with CRC (as noted in the second and fourth groups) were grouped with patients without CRC. Interesting, none of these ectopic CRC patients had hepatic metastases. Thus, according to yet another aspect of the present invention, manifestations of clustering provide a basis for early subclinical detection and pretreatment of occult CRC in patients lacking clinical symptoms.

An unsupervised hierarchical cluster analysis was performed to determine whether aggregation of genes by their expression similarity level per patient contributed to segregation of patients with and without CRC. Application of Euclidean distances between studied genes resulted in the appearance of clusters allowing the distribution of patients according to their transcriptional similarity levels. As shown in FIG. 11A, the heatmap outlined four mixed subgroups of patients with distinct gene expression patterns, two of them enriched by patients with CRC and two others by patients without CRC. A new heatmap (FIG. 11B) was constructed using genes with the highest predictive power of the class of patient, as evidenced in the PLS-DA analysis. In this case, the power of discrimination was comparable to that obtained in the previous heatmap, but in this case there was a segregation in two large mixed subgroups rather than four, both of which being enriched either in patients with CRC or without CRC. Some patients without CRC are grouped with patients with CRC suggesting they may have occult CRC (which was later confirmed by colonoscopy), while some patients with CRC are grouped with patients without CRC (none of CRC patients had hepatic metastases).

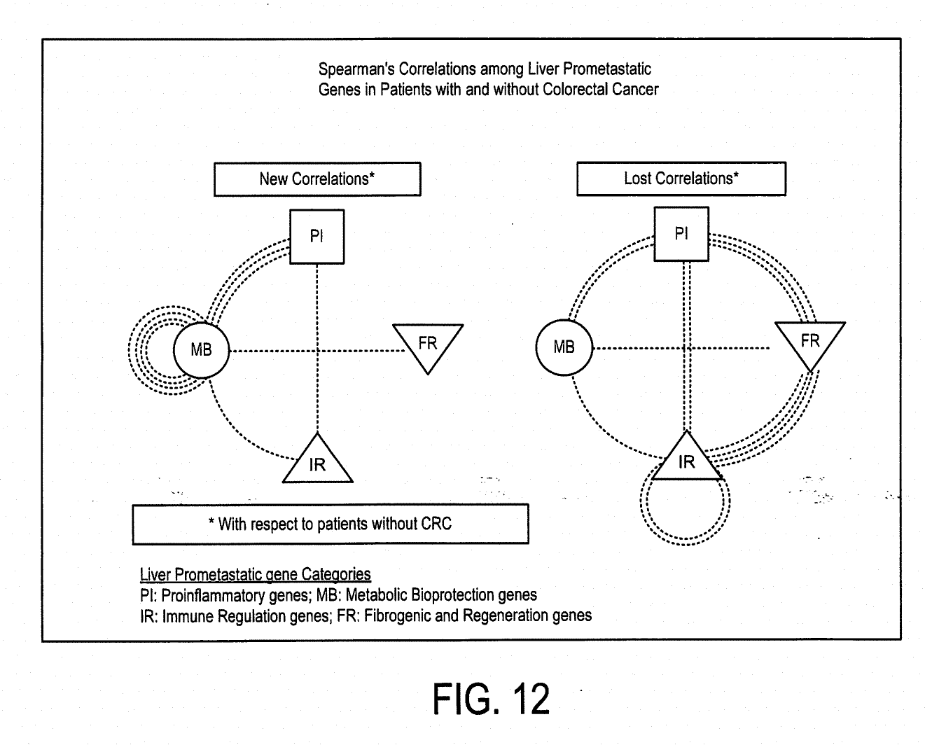

FIGS. 12A and 12B show Spearman's correlation of expression levels among liver prometastatic genes in patients with and without CRC. Only statistically significant (p<0.05 or higher) correlations with coefficient Rho equal to or greater than 0.7 were considered in this analysis. Nine of the ten correlations in patients with CRC involved five bioprotective genes (HP, ERBB2IP, GAPDH, CRP, PDRX4); four of these correlation gains were produced among bioprotective genes (ERBB2IP-GAPDH; ERBB2IP-PDRX4; CRP-GAPDH; CRP-HP), three among metabolic bioprotection and proinflammatory genes (CRP-TNFSF14; HP-TNFSF14; GAPDH-ID1) (CRP-NGF), immune-regulation genes (PDRX4-CEACAM1), as well as between proinflammatory and immune-regulation genes (TNFSF14-COL18A1). In contrast, eight out of the fourteen lost gene correlations in patients with CRC occurred in immune-regulation gene group (involving CEACAM1, MRC1, ICAM1, IL10, BMP7 genes), of which four were lost between immune-regulation and fibrogenic/regeneration genes (ICAM1-TGFB1; IL-10-NGF, CEACAM1-NGF, MRC1-NGF), whereas only two were lost between immune-regulation and proinflammatory genes (BMP7-TNF, CEACAM1-TNF) and another two among immune-regulation genes (CEACAM1-BMP7; MRC1-BMP7). There was also a striking loss of seven correlations between proinflammatory genes and other functional categories of genes (metabolic bioprotection, fibrogenic/regeneration and immune-regulation genes). Thus, in yet another aspect of the present invention, a statistically significant manifestation of a number and/or functional category of lost and new correlations of gene expression levels relative to such correlations in patients without CRD provides an additional rule-based system (the multigenic inter- and intra-functional group transcriptional relationships) and/or methodology to predict and treat occult CRC in patients lacking evidence of clinical symptoms.

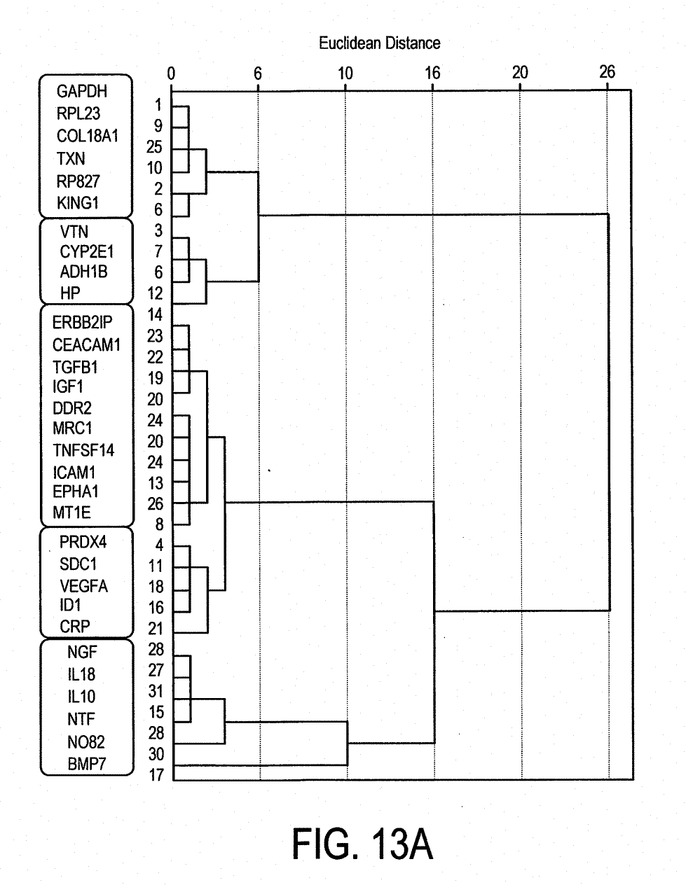

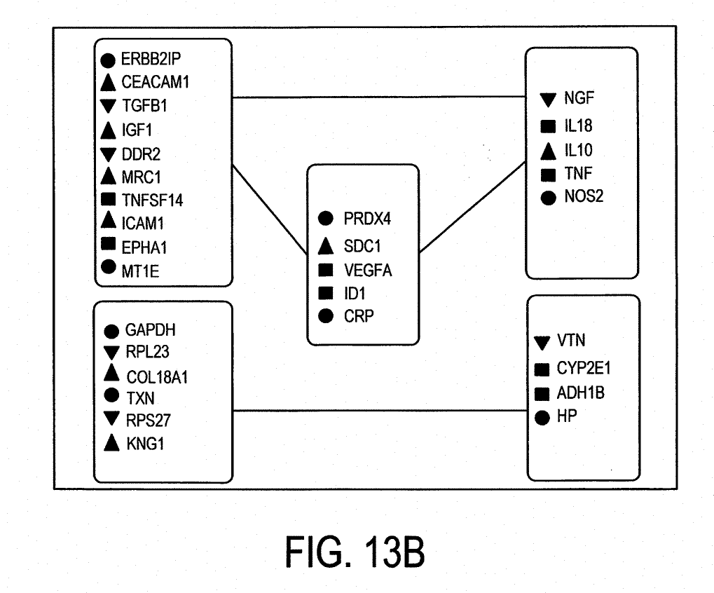

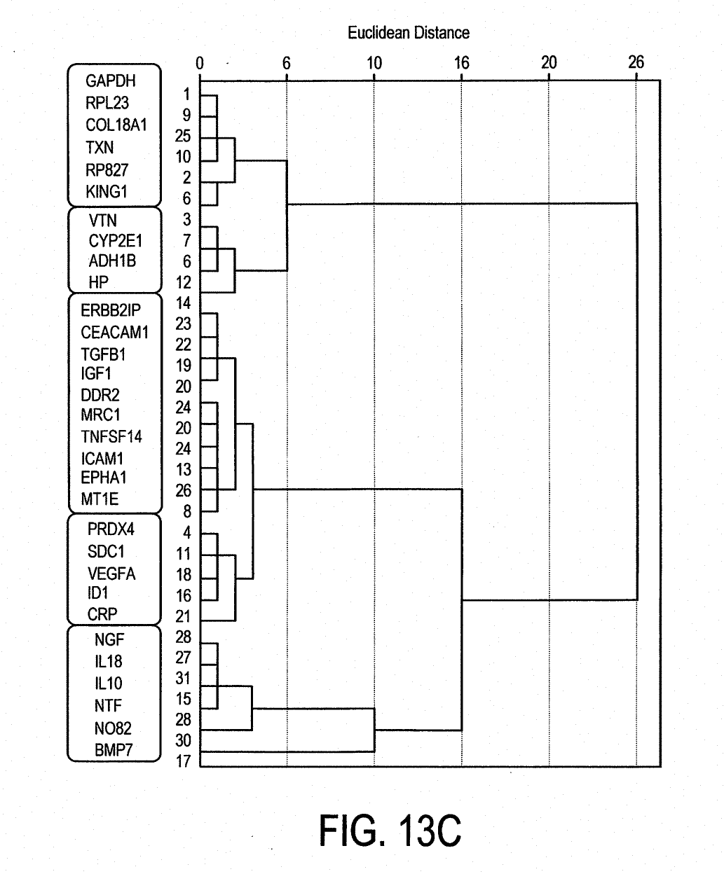

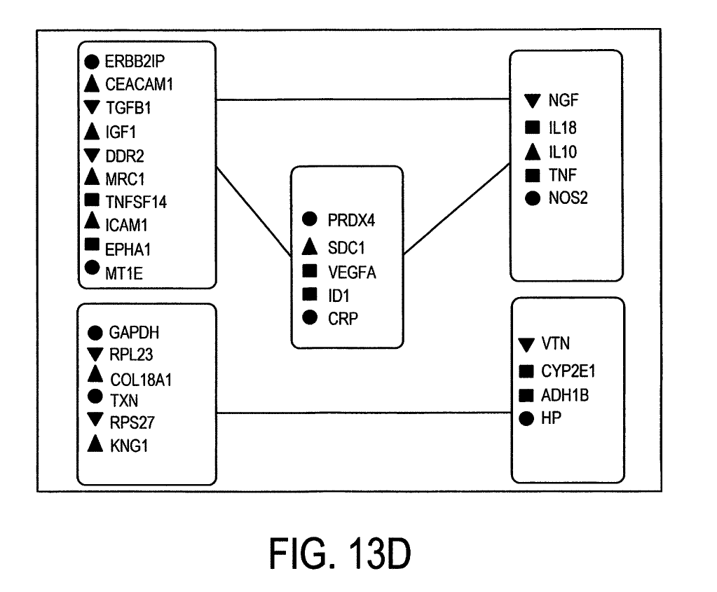

FIG. 13, Charts A-D, show hierarchical clustering performed based on Pearson's correlation Euclidean distance among the genes and gene clusters, and the results presented as a dendrogram plot in order to define the transcriptional structure of prometastatic genes in hepatic biopsies from patients without (Chart A) and with (Chart C) CRC. A cluster primarily including PRX4, SDC1, VEGFA, ID1 and CRP genes define the main change in the hepatic transcriptional structure between patients without and with CRC, as shown in Charts B and D. This analysis may be automated using data processing device or equipment. Thus, according to another aspect of the invention, hierarchical clustering of PRX4, SDC1, VEGFA, ID1 and CRP genes may form a subclinical parameter or indicator that is utilized by a data processing device to systematically automate prediction of CRC cancer risk and provide an alert of possible occult CRC in patients without clinical evidence of CRC but with other diseases that increase CRC risk.

Spearman's correlation analysis was used to study the structure of transcriptional associations among liver prometastatic genes in patients with and without CRC, and to identify those gene correlations changing between patients with and without CRC. As shown in FIG. 12, correlations among genes from patients with CRC were strengthened in the metabolic bioprotection gene group, while they were lost among genes in the immune-regulation gene group. A hierarchical clustering (FIG. 13) was performed based on Pearson's correlation Euclidean distances among liver prometastatic genes and their gene dusters, and represented as dendrogram plots in order to define the transcriptional structure of prometastatic genes in hepatic biopsies from patients with and without CRC (FIG. 13, Charts A and C). A cluster including PRX4, SDC1, VEGFA, ID1 and CRP genes defined the main change in the hepatic transcriptional structure between patients with and without CRC (Charts C and D). Therefore, an additional feature contributing to identifying CRC-dependent gene expression changes in patients without clinical evidence of CRC is the correlation pattern among liver prometastatic genes.

It was also revealed that the relationship between and among gene expression levels within functional categories differ according to location of the primary tumor in patients having CRC cancer. According to another aspect of the present invention, the inventive device may determine and use this information to identify or direct a type of treatment administered to a patient. FIGS. 4A, 4B, 4C and 4D, for example, show differences in distributions of liver prometastatic genes in high-expressing patients according to whether the primary tumor is located in the rectum (dotted trace), right-side (dot-dash trace) colon and left-side (dashed trace) colon. In particular, FIG. 4A shows a first relational distribution of liver prometastatic gene expressions in high-expressing patients for Proinflammatory genes (IL18, ID1, TNF, VEFA, EPHA1, TSFSF14, CYP2E1 and ADH1B) according to primary tumor location in patients with CRC. FIG. 4B shows a second relational distribution of liver prometastatic gene expressions in high-expressing patients for Immune regulation genes (ICAM1, IL10, MRC1, KNG1, SDI1, COL18A1, IGF1 and MP 7) according to primary tumor location in patients with CRC. FIG. 4C shows a third relational distribution of liver prometastatic gene expressions in high-expressing patients for Metabolic Bioprotection genes (GAPDH, PRDX4, TXN, MT1E, HP, NOS2, CRP, and ERBB2IP) according to primary tumor location in patients with CRC. FIG. 4D shows a fourth relational distribution of liver prometastatic gene expressions in high-expressing patients for Fibrogenic/Regeneration genes (RPL23, DDR2, TGFB1, VTN and NGF) according to primary tumor location in patients with CRC.

A further aspect of the inventive apparatus includes a complementary diagnostic test to indicate a possible anatomical location of an occult CRC in patients without clinical evidence of CRC, but with other digestive system diseases increasing CRC risk, such as cholelithiasis and metabolic syndrome. As shown in FIGS. 13 and 4A-4D, patients with tumors of localization in the left-side colonic area (including splenic flexure, descending colon, sigmoid colon or recto sigmoid junction) were the ones that most frequently increased the expression of liver prometastatic genes, followed by patients with right-sided tumors (including cecum, ascending colon, hepatic flexure or transverse colon), whereas patients with rectal tumors were those more frequently decreasing the expression level. The anatomical location of CRC determined the liver prometastatic gene expression pattern and the percentage of patients with high and low expression of these genes. Therefore, in accordance with this further aspect of the invention, the inventive device may develop and use these patterns suggest the possible anatomical location of an occult CRC in patients without clinical evidence of CRC, but with other digestive system diseases increasing CRC risk, such as cholelithiasis and metabolic syndrome in order to provide a basis to direct and determine a best possible treatment regime

Table 6 shows distribution of liver prometastatic genes by functional categories and tumor location. Rectal Tumor Pattern is indicate by Low hepatic expression of genes from the four prometastatic gene functional categories with high-IL10, MRC1 and NOS2 gene expression, which suggest Immunotolerance/immunosuppression without inflammatory background and possible beneficial effects of immunotherapy in metastasis prevention. Left-sided colonic Tumor Pattern (including CRC within the splenic flexure, descending colon, sigmoid colon or recto sigmoid junction) is indicated by High hepatic expression of proinflammatory, immune regulation and metabolic bioprotection genes, with drop in BMP7 and NGF gene expression, which suggests very high-risk prometastatic microenvironment and possible beneficial effects of anti-inflammatory therapies in metastasis prevention. Right-sided colonic Tumor Pattern (including primary CRC in the cecum, ascending colon, hepatic flexure or transverse colon) is indicated by a slight increase of proinflammatory and immune regulation gene expression with ADH1B, SDC1 and VT gene expression decrease, which suggests slight immunotolerance/immunosuppression under inflammatory conditions and possible beneficial effect of anti-inflammatory therapies in metastasis prevention. According to yet another aspect of the invention, the apparatus may perform an analysis to determine a treatment regime in accordance with high-low gene expression levels of genes within respective functional categories and anatomic location of the tumor along the colonic tract. Such a processing device or apparatus may provide such determination in an automated diagnostic and treatment system.

Personalized treatment of patients based on a multiplex of molecular biomarkers defining precise functional features of cancer that may strongly increase the efficacy of the chosen therapies. In this study, the analysis of liver prometastatic gene functional categories by anatomical location of the CRC identified three distinct functional patterns with therapeutic implications (Table 6 and FIG. 4). Rectal Tumor Pattern was indicated by Low hepatic expression of genes from the four prometastatic gene functional categories with high-IL10, MRC1 and NOS2 gene expression, which suggest Immunotolerance/immunosuppression without inflammatory background and possible beneficial effects of immunotherapy in metastasis prevention. Left-sided colonic Tumor Pattern is indicated by High hepatic expression of proinflammatory, immune regulation and metabolic bioprotection genes, with drop in BMP7 and NGF gene expression, which suggests very high-risk prometastatic microenvironment and possible beneficial effects of anti-inflammatory therapies in metastasis prevention. Right-sided colonic Tumor Pattern was indicated by Slight increase of proinflammatory and immune regulation gene expression with ADH1B, SDC1 and VT gene expression decrease, which suggests slight Immunotolerance/immunosuppression under inflammatory conditions and possible beneficial effect of anti-inflammatory therapies in metastasis prevention.

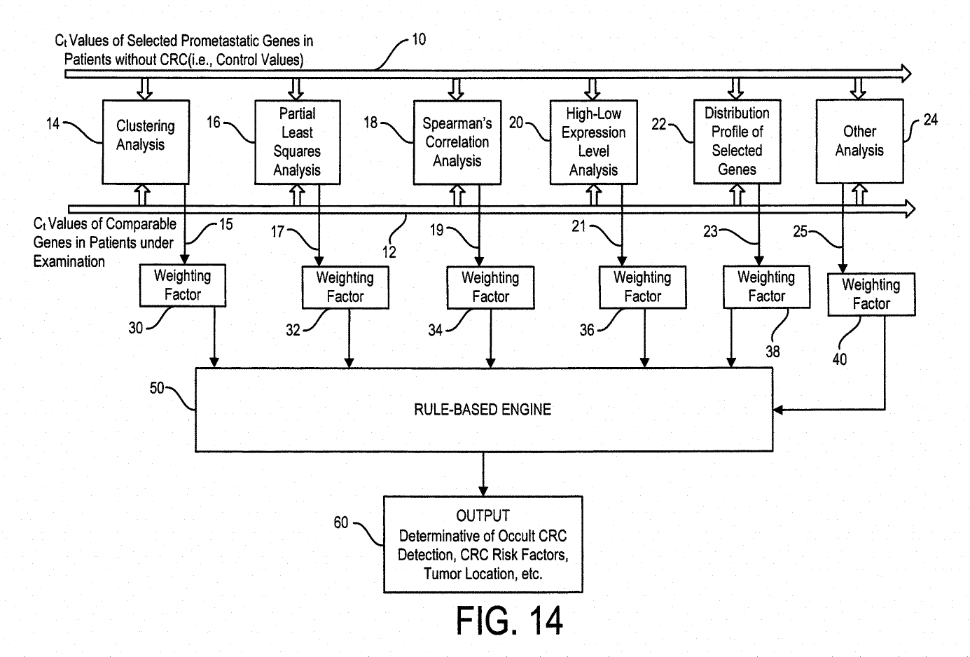

FIG. 14 shows an exemplary apparatus that processes gene expression data to determine CRC risks and/or to determine a proposed treatment according to the present invention where input 10 receives gene expression levels (i.e., Cₜ values is as control levels) of patients without CRC and input 12 receives gene expression level of a patient under examination who may or may not have a CRC risk. Module 14 performs clustering analysis, module 16 performs a partial least squares analysis, module 18 performs a Spearman's or other correlation analysis, module 20 assess over-expressed and under-expressed gene expression levels (i.e., high-low express levels), module 22 performs a distribution analysis, and module 24 performs other analysis indicative of a divergent gene expression levels of the patient under examination. The rule-based device processes gene express levels in a series of program modules 14, 16, 18, 20, 22 and 23 according to the respective analysis. Module 14, for example, executes program instructions to perform a clustering analysis of expression levels of the patient under examination from input 12 against control levels supplied from input 10.

An initial series of computational results of expression level processing from respective modules 14-24 may be weighted by predetermined weighting factors 30-40 according to their significance. For example, if different weights are to be assigned to computational results of the respective modules, results of each module may be assigned a weighting factor between 0.5 and 1.0 according to their importance. Such weighting factors may be assigned by a testing laboratory, researcher, medical practitioner, or may simply predetermined as a fixed value. Control or reference values against which patient data is compared may be fixed, or determined impromptu when obtaining patient data. A clustering analysis may be assigned a weighting factor higher or lower than other that other modules. After applying any such weighting factor, the results from modules 14-24 are then supplied to a rule-based engine 50 to produce final results taking into consideration all gene expression data, which then products an output at 60 deterministic of occult CRC, CRC risk factors, tumor location, etc.

The written description, drawing figures, tables and charts presented herein are not intended to limit the scope of the invention but merely provide an illustration of the core concepts and embodiments that may be implemented to carry out the teachings set forth herein. Based on these teachings, persons skilled in the art may devise alternative embodiments or modify the illustrated embodiments without departing from the scope of the invention. Accordingly, the scope of invention is defined by the appended claims rather than by the description or illustrated embodiments.

### REFERENCES

Anasagasti M J, Alvarez A, Mendoza L, Martin J J y Vidal-Vanaclocha F. Sinusoidal endothelium release of hydrogen peroxide enhances VLA-4 mediated melanoma cell adherence and tumor cytotoxicity during interleukin-1 promotion of melanoma hepatic metastasis. Hepatology 25:840-846 (1997).

Anasagasti M J, Martin J J, Mendoza L, Obrador E, Estrela J M, McCuskey R S, and Vidal-Vanaclocha F. Glutathione protects metastatic melanoma cells against oxidative stress in the murine hepatic microvasculature. Hepatology 27: 1249-1256 (1998).

Anasagasti M J, Olaso E, Calvo F, Martin J J, Mendoza M, Bidaurrazaga J, and Vidal-Vanaclocha F. Interleukin-1 (IL-1)-Dependent and -Independent Mouse Melanoma Metastases. J Natl Cancer Institute 89:645-651 (1997).

Aoyama T, Kashiwabara K, Oba K, Honda M, Sadahiro S, Hamada C, Maeda H, Mayanagi S, Kanda M, Sakamoto J, Saji S, Yoshikawa T. Clinical impact of tumor location on the colon cancer survival and recurrence: analyses of pooled data from three large phase III randomized clinical trials. Cancer Med. 2017 Sep 25. doi: 10.1002/cam4.1208.

Arteta B, Lasuen N, Sveinbjornssøn B, Smedsrød B and Vidal-Vanaclocha F. Murine Colon Carcinoma Cell interaction with Liver Sinusoidal Endothelium Inhibits Anti-Tumor Immunity via IL-1 Induced Mannose Receptor. Hepatology 2010; 51: 2172-2182.

Arteta B, Lasuen N, Sveinbjornssøn B, Smedsrød B and Vidal-Vanaclocha F. Murine Colon Carcinoma Cell interaction with Liver Sinusoidal Endothelium Inhibits Anti-Tumor Immunity via IL-1 Induced Mannose Receptor. Hepatology 2010; 51: 2172-2182.

Badiola I, Olaso E, Crende O, Friedman S L, Vidal-Vanaclocha F. Discoidin domain receptor 2 deficiency predisposes hepatic tissue to colon carcinoma metastasis. Gut. 2012 October;61(10):1465-72.

Badiola I, Olaso E, Crende O, Friedman S L, Vidal-Vanaclocha F. Discoidin domain receptor 2 deficiency predisposes hepatic tissue to colon carcinoma metastasis. Gut. 2012 October;61(10):1465-72.

Barbois S, Arvieux C, Leroy V, Reche F, Stürm N, Borel AL. Benefit-risk of intraoperative liver biopsy during bariatric surgery: review and perspectives. Surg Obes Relat Dis. 2017 Aug. 14. pii: S1550-7289(17)30359-3.

Beaskoetxea J ; Ruiz-Casares E; Telleria N; del Villar A; Garcia de Durango C; Lapuente F; Gil A; Fernandez-Nespral V; Ielpo B; Carusso R; Duran H; Quijano Y; de Vicente E; Vidal-Vanaclocha F. Liver metastasis-Associated colon cancer cell genes: microenvironmental regulation and therapeutic implications. J Hepatology (in preparation).

Carrascal T, Mendoza L, Vacarcel M, Salado C, Egilegor E, Telleria N, Vidal-Vanaclocha F and Dinarello C A. Interleukin/18 binding protein reduces B16 Melanoma Hepatic Metastasis by neutralizing the adhesiveness and growth factors of sinusoidal endothelial cell. Cancer Res 63:491-7 (2003)

Crende O, Sabatino M, Valcárcel M, Carrascal T, Riestra P, López-Guerrero JA, Nagore E, Mandruzzato S, Wang E, Marincola FM, Vidal-Vanaclocha F. Metastatic lesions with and without interleukin-18-dependent genes in advanced-stage melanoma patients. Am J Pathol. 2013 July;183(1):69-82.

García de Durango C, Marina Pérez-Gordo; Eva Ruiz Casares, and Fernando Vidal-Vanaclocha. Transcriptional Association of Bacterial Endotoxin-Dependent Colorectal Cancer Cell Soluble Proteins to Hepatic Prometastatic Signature Genes. Gastroenterology (in preparation).

García De Durango C r, De Wit M, Piersma S r, Knol J, Pham T v, Pérez-Gordo M, Fijneman R j a, Vidal-Vanaclocha F, Jimenez C r. Lipopolysaccharide-regulated secretion of soluble and vesicle-based colorectal cancer cell proteins. J Proteomics 2017 (in preparation).

Gil A; García de Durango C; Lapuente F; Fernandez-Nespral V; Ielpo B; Carusso R; Duran H; Quijano Y; de Vicente E; Vidal-Vanaclocha F. Pathophysiological anthropometric correlations of liver prometastatic genes in patients with primary colorectal cancer. Am J Pathol (in preparation).

Gosavi S, Mishra R R, Kumar V P. Study on the Relation between Colorectal Cancer and Gall Bladder Disease. J Clin Diagn Res. 2017 March;11(3):0C25-0C27.

Lee T, Yun K E, Chang Y, Ryu S, Park D I, Choi K, Jung Y S. Risk of Colorectal Neoplasia According to Fatty Liver Severity and Presence of Gall Bladder Polyps. Dig Dis Sci. 2016 January;61(1):317-24.

Márquez J, Kohli M, Arteta B, Chang S, Li W B, Goldblatt M, Vidal-Vanaclocha F. Identification of hepatic microvascular adhesion-related genes of human colon cancer cells using random homozygous gene perturbation. Int J Cancer. 2013 Nov;133(9):2113-22.

Marquez J, Kohli M, Arteta B, Chang S, Li WB, Goldblatt M, Vidal-Vanaclocha F. Identification of hepatic microvascular adhesion-related genes of human colon cancer cells using random homozygous gene perturbation. Int J Cancer. 2013 November;133(9):2113-22.

Marshall J C, Collins J W, Nakayama J, Horak C E, Liewehr D J, Steinberg S M, Albaugh M, Vidal-Vanaclocha F, Palmieri D, Barbier M, Murone M, Steeg P S. Effect of inhibition of the lysophosphatidic acid receptor 1 on metastasis and metastatic dormancy in breast cancer. J Natl Cancer Inst. 2012 Sep. 5;104(17):1306-19.

Mendoza L, Carrascal T, De Luca M, Fuentes A M, Salado C, Blanco J, Vidal-Vanaclocha F. Hydrogen peroxide mediates vascular cell adhesion molecule-1 expression from interleukin-18-activated hepatic sinusoidal endothelium: implications for circulating cancer cell arrest in the murine liver. Hepatology 34:298-310 (2001).

Mendoza L, Valcarcel M, Carrascal T, Egilegor E, Salado C, Sim B K, Vidal-Vanaclocha F. Inhibition of cytokine-induced microvascular arrest of tumor cells by recombinant endostatin prevents experimental hepatic melanoma metastasis. Cancer Res 64: 304-10 (2004)

Olaso E, Salado C, Gutierrez V, and Vidal-Vanaclocha F. Proangiogenic Role of Tumor-Activated Hepatic Stellate Cells in Melanoma Metastasis. Hepatology 37:674-85 (2003)

Olaso E, Santisteban A, Bidaurrazaga J, Gressner A M, Rosenbaum J, and Vidal-Vanaclocha F. Tumor-dependent activation of hepatic stellate cells during experimental melanoma metastasis. Hepatology 26: 634-642 (1997).

Ruiz-Casares E; Lapuente F; Ielpo B; Carusso R; Duran H; Quijano Y; de Vicente E; Vidal-Vanaclocha F. Prometastatic gene expression patterns in the liver of patients with and without colorectal cancer: pathogenic implications and clinical correlations. Hepatology (in preparation)

Solaun M S, Mendoza L, de Luca M, Gutierrez V, Lopez M-P, Olaso E, B, Sim B K L, Vidal-Vanaclocha F. Endostatin Inhibits Murine Colon Carcinoma Sinusoidal-Type Metastases by Preferential Targeting of Hepatic Sinusoidal Endothelium. Hepatology 35: 1104-1116 (2002)

Valcárcel M, Carrascal T, Crende O, Vidal-Vanaclocha F. IL-18 Regulates Melanoma VLA-4 Integrin Activation through a Hierarchized Sequence of Inflammatory Factors. J Invest Dermatol. 2014 February;134(2):470-80.

Vidal-Vanaclocha F, Fantuzzi G, Mendoza L, Fuentes A M, Anasagasti M J, Martin J J, Carrascal T, Walsh P, Reznikov L L, Kim S-H, Novick D, Rubinstein M, and Dinarello C A. IL-18 regulates IL-1beta-dependent hepatic melanoma metastasis via vascular cell adhesion molecule-1. Proc Nat Acad Sci USA 97: 734-39 (2000).

Vidal-Vanaclocha F, Mendoza L, Telleria N, Salado C, Valcarcel M, Gallot N, Carrascal T, Egilegor E, Beaskoetxea J, Dinarello. Clinical and experimental approaches to the pathophysiology of interleukin-18 in cancer progression. Cancer Metastasis Rev 25:417-34 (2006)

Vidal-Vanaclocha F. Architectural and Functional Aspects of the Liver with Implications for Cancer Metastasis. P. Brodt (ed.), Liver Metastasis: Biology and Clinical Management, Cancer Metastasis—Biology and Treatment 16, DOI 10.1007/978-94-007-0292-9_2, C_{—} Springer Science+Business Media B.V. 2011a.

Vidal-Vanaclocha F. Regulation of Liver Metastasis-Related Genes at Primary and Metastatic Tumors in the Pathophysiological Context of the Colorectal Cancer Disease. Gut (review article in preparation).

Vidal-Vanaclocha F. The Liver Prometastatic Reaction of Cancer Patients: Implications for Microenvironment-Dependent Colon Cancer Gene Regulation. CAMI 2011;4(2):163-80.

Vidal-Vanaclocha F. The prometastatic microenvironment of the liver. Cancer Microenvir, 2008; 1: 113-129

Vidal-Vanaclocha F. The Tumor Microenvironment at Different Stages of Hepatic Metastasis P. Brodt (ed.), Liver Metastasis: Biology and Clinical Management, Cancer Metastasis—Biology and Treatment 16, DOI 10.1007/978-94-007-0292-9_3, C_{—} Springer Science+Business Media B.V. 2011 b.

Yahagi M, Okabayashi K, Hasegawa H, Tsuruta M, Kitagawa Y. The Worse Prognosis of Right-Sided Compared with Left-Sided Colon Cancers: a Systematic Review and Meta-analysis. J Gastrointest Surg. 2016 March;20(3):648-55.

Zhao X, Li L, Starr T K, Subramanian S. Tumor location impacts immune response in mouse models of colon cancer. Oncotarget. 2017 Jun. 9;8(33):54775-54787.

I claim:

1. A rule-based apparatus to detect metastatic cancer in a patient having a colorectal tumor, said apparatus comprising:
(a) an input to receive patient data comprising a plurality of genetic expression levels of genes from tumor unaffected hepatic tissue of the patient, wherein said genes include selected ones of genes from group 1 genes (PRDX4, CRP, ID1, MT1 E, TNFSF14, MRC1, ICAM1, IL18, IL10, TFN) and group 2 genes (NGF, EPHA1, ERBB2IP, SDC1, COL18A1, KNG1, ADH1B, CYP2E1);
(b) a memory that stores respective reference values for group 1 and group 2 genes wherein said reference values respectively indicate expression levels of corresponding genes of a person free of colorectal tumors;
(c) a processor responsive to said patient data and said reference values to interpret significance of overexpression of selected group 1 patient data genes relative to group 1 reference values and/or significance of underexpression of selected group 2 patient data genes relative to group 2 reference values; and
(d) an output responsive to said processor to produce an indication confirming or annulling hepatic metastasis according to interpretation of significance of overexpression and/or underexpression of group 1 and/or group 2 patient data genes.

2. The apparatus of claim 1, wherein processor assigns a weight to respective selected genes according to predetermined significance of indication of hepatic metastasis.

3. The apparatus claim 1, wherein said processor utilizes protein signatures of said genes to indicate overexpressions or underexpressions thereof.

4. The apparatus of claim 1, wherein said processor utilizes correlation, clustering and/or heatmaps to interpret significance of said gene expressions.

5. The apparatus of claim 1, wherein said processor additionally utilizes selected ones of group 3 genes (HP, VTN, RPS27, RPL23, GAPDH, TXN, VEGFA, CEACAM1, IGF1, TGFB1, DDR2, NOS2, and BMP7) to validate significance of gene expressions.

6. The apparatus of claim 5, wherein selected ones of genes comprises statistically significant ones of group 1, group 2 and group 3 genes.

7. The apparatus of claim 1, wherein said processor comprises a digital microprocessor.

8. The apparatus of claim 1, wherein said patient data and reference values comprise cycle count information derived from polymerase chain reactions.

9. The invention of claim 1, further comprising a kit to derive patient data, wherein the kit comprises (a) instructions for performing gene assessments and (b) either a low density genetic expression array of reagents for PCR replication/detection or a low density protein array of antibodies for hybridization with patient's blood serum/plasma.

10. The apparatus of claim 1, wherein said processor detects a left-side colon location of a CRC tumor according to overexpressed levels of statistically significant ones of (i) proinflammatory genes IL18, ID1, TNF, TNFSF14, AND ADH1B, (ii) immune regulation genes ICAM1, MRC1, KNG1, and SDC1, and/or (iii) metabolic bioprotection genes PRXD4, MTE1, P, NOS2 and CRP.

11. The apparatus of claim 1, wherein said processor further detects a rectal location of a CRC tumor according to underexpressed levels of statistically significant ones of (i) IL18, ID1, VEGFA, TNFSF14, ADH1B and CYP2E1 proinflammatory genes, (ii) ICAM1, KNG1, SDC1 AND BMP7 immuno regulation genes, and (iii) GAPDH, TXN, MTE1, HP, CR AND ERBB2IP metabolic bioprotection genes.

12. The apparatus of claim 1, wherein said processor detects a right side colon location of a CRC tumor according to (i) high expression level of at least one of ID1 and TNF proinflammatory genes, (ii) low expression level of at least one of ADH18 and CYPE1 proinflammatory genes, (iii) high expression level of at least one of immune regulation genes IL10, MRC1 and BMP7, (iv) low expression level of at least one of immune regulation genes KNG1 and SDC1, and (v) low expression level of at least one of VTN and NGF fibrogenic and regeneration genes.

13. The apparatus of claim 1, wherein said processor comprises a series of software modules to execute program instructions to perform at least two of (i) a partial least squares-discriminant analysis of said selected genes of a patient with and a patient without CRC, (ii) a clustering analysis of selected genes in a patient with and a patient without CRC, (iii) a Spearman's correlation analysis of selected genes to assess new and lost correlations of selected genes in respective categories in a patient with and a patient without CRC, (iv) a hierarchical clustering analysis of selected genes in a patient with and a patient without CRC, (v) distribution analysis of selected gene expression levels for genes in respective functional categories in a patient with and a patient without CRC, and (vi) determining high-low expression levels of selected genes in functional categories indicative of location of primary tumors in a patient with and a patient without CRC.

14. A rule-based apparatus to detect occult cancer in a target patient having a gastrointestinal disorder, said apparatus comprising:
(a) an input to receive patient data comprising a plurality of genetic expression levels of genes from tumor unaffected hepatic tissue of the patient, wherein said genes include selected ones of genes from group 1 (PRDX4, CRP, ID1, MT1 E, TNFSF14, MRC1, ICAM1, IL18, IL10, TFN) and/or group 2 (NGF, EPHA1, ERBB2IP, SDC1, COL18A1, KNG1, ADH1B, CYP2E1);
(b) a memory that stores respective reference values for group 1 and group 2 genes wherein said reference values indicate respective expression levels of corresponding genes of a person free of colorectal tumors;
(c) a processor responsive to patient data and said reference values (i) to interpret significance of overexpression of said selected group 1 patient data genes relative to said group 1 reference values and/or underexpression of selected group 2 patient data genes relative to said group 2 reference values; and
(d) an output responsive to said processor to produce an indication confirming or denying hepatic metastasis according to interpretation of significance of overexpression and/or underexpression of group 1 and/or group 2 patient data genes.

15. The invention of claim 14, wherein said processor additionally utilizes selected ones of group 3 genes (HP, VTN, RPS27, RPL23, GAPDH, TXN, VEGFA, CEACAM1, IGF1, TGFB1, DDR2, NOS2, and BMP7) to validate significance of gene expressions of group 1 and group 2 genes.

16. A kit to assist a user to produce patient data for detecting hepatic metastasis, said kit comprising (a) a genetic expression array for measuring genetic expressions of group 1 genes (PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1B, CYP2E1), and/or group 3 genes (HP, VTN, RPS27, RPL23, GAPDH, TXN, VEGFA, CEACAM1, IGF1, TGFB1, DDR2, NOS2, and BMP7) and (b) instructions for using the genetic expression array.

17. The kit of claim 16, wherein said genetic expression array comprises a low density genetic expression array.

18. A kit to assist a user to produce patient data for detecting hepatic metastasis, said kit comprising (a) a protein array for measuring protein signatures of group 1 genes (PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1B, CYP2E1), and/or group 3 genes (HP, VTN, RPS27, RPL23, GAPDH, TXN, VEGFA, CEACAM1, IGF1, TGFB1, DDR2, NOS2, and BMP7) and (b) instructions for using the protein array.

19. The kit of claim 18, wherein said protein array comprises a low density protein array.
